(19) United States
(12) Patent Application Publication
(10) Pub. No.: US 2025/0322917 A1
(43) Pub. Date: Oct. 16, 2025

(54) UTILIZING FLOW MEASURES OF A GENERATIVE STOCHASTIC MODEL AND ACTION VALUES OF AN ACTION-VALUE MODEL TO GENERATE STRUCTURAL REPRESENTATIONS

(71) Applicant: Recursion Pharmaceuticals, Inc., Salt Lake City, UT (US)

(72) Inventors: Emmanuel Bengio, Montreal (CA); Tsoi Yung Lau, Jersey City, NJ (US)

(21) Appl. No.: 18/633,693

(22) Filed: Apr. 12, 2024

(51) Int. Cl. G16C 20/50

(52) U.S. Cl. CPC G16C 20/50

(57) ABSTRACT

The present disclosure relates to systems, non-transitory computer-readable media, and methods that utilize a generative stochastic model and an action-value function model to build a biochemical structure. Indeed, in one or more implementations, the disclosed systems generate a flow measure for a constructive object option in building a biochemical structure and further generate an action-value for the constructive object option. For instance, the disclosed systems combine the flow measure and the action-value to select the constructive object option from a plurality of constructive object options. Moreover, in some instances, the disclosed systems generate the biochemical structure using the selected constructive object option.

### BACKGROUND

Recent years have seen significant developments in hardware and software platforms for training and utilizing generative methods to explore complex feature spaces. For example, conventional systems train generative methods to diversely sample complex structures such as molecular compounds. Despite these recent advances, conventional systems suffer from a number of technical deficiencies, particularly with regard to accuracy, efficiency, and operational inflexibility in exploring and generating structures in complex feature spaces.

### SUMMARY

Embodiments of the present disclosure provide benefits and/or solve one or more of the foregoing or other problems in the art with systems, non-transitory computer-readable media, and methods for generating novel biological or chemical structures utilizing a generative machine learning framework that utilizes flow measures of generative stochastic model and action values of an action-value model. For example, to generate biochemical structures, the disclosed systems combine a flow measure with an action-value estimate (e.g., Q) to create improved sampling policies which can be controlled by a mixing hyperparameter. Specifically, the disclosed systems utilize a combination of the outputs from a generative stochastic model (e.g., a generative flow network) and an action-value function model to improve on exploring the number of high-reward objects without sacrificing diversity. For instance, the disclosed systems utilize a combination of an action-value estimate and a flow measure to iteratively select constructive object options in building a novel biological or chemical structure.

Additional features and advantages of one or more embodiments of the present disclosure are outlined in the description which follows, and in part will be obvious from the description, or may be learned by the practice of such example embodiments.

### BRIEF DESCRIPTION OF THE DRAWINGS

The detailed description provides one or more embodiments with additional specificity and detail through the use of the accompanying drawings, as briefly described below.

FIG. 1 illustrates an overview diagram of a QGFN generation system adding a constructive object option to generate a biochemical structure in accordance with one or more embodiments.

FIG. 2 illustrates an example diagram of the QGFN generation system selecting a constructive object option from a plurality of constructive object options in accordance with one or more embodiments.

FIG. 3 illustrates an example diagram of the QGFN generation system performing an additional construction stage in accordance with one or more embodiments.

FIG. 4 illustrates an example diagram that compares a flow measure with an action-value in accordance with one or more embodiments.

FIG. 5 illustrates an example diagram of the QGFN generation system utilizing a variety of approaches to combine a flow measure and an action-value in accordance with one or more embodiments.

FIG. 6 illustrates an example diagram of the QGFN generation system training a generative stochastic model and an action-value function model in accordance with one or more embodiments.

FIGS. 7A-7B illustrates experimental results of adjusting a beta hyperparameter to prioritize reward and the effect of adjusting a p-value to prioritize reward in accordance with one or more embodiments.

FIGS. 8A-8B illustrates experimental results of a variety of methods for mixing an action-value and a flow measure and their effectiveness with respect to particular tasks (e.g., molecular generation task and RNA generation task) in accordance with one or more embodiments.

FIGS. 9A-9B illustrates experimental results for trade-offs between diversity and reward for a variety of methods and comparing action-value predictions with empirical estimates in accordance with one or more embodiments.

FIG. 10 illustrates experimental results of the QGFN generation system and masking according to action-value in accordance with one or more embodiments.

FIG. 11 illustrates an example environment of the QGFN generation system in accordance with one or more embodiments.

FIG. 12 illustrates an example series of acts to generate a biochemical structure utilizing a constructive object option in accordance with one or more embodiments.

FIG. 13 illustrates a block diagram of a computing device for implementing one or more embodiments.

### DETAILED DESCRIPTION

Embodiments of the present disclosure provide benefits and/or solve one or more of the foregoing or other problems in the art with systems, non-transitory computer-readable media, and methods for generating novel biological or chemical structures utilizing a generative machine learning framework. For example, the QGFN generation system combines a generative stochastic model (e.g., a generative flow network) with an action-value function model (e.g., Q) to improve sampling policies and generate additional high-reward objects in a variety of tasks without sacrificing diversity. Specifically, the QGFN generation system utilizes the generative stochastic model to generate objects, (e.g., such as biochemical structures) by modeling flow into possible downstream paths, where the measure of flow is proportionate to the cumulative probability of reward for each path. In some embodiments, the QGFN generation system utilizes the generative stochastic model to build a biochemical structure by sequentially adding a next component based on the highest measure of flow for available paths. As such, relying on just the generative stochastic model often leads to emphasizing exploratory paths (i.e., the model often chooses paths with a high cumulative possibility of reward, even though any particular final result within a path has a relatively low reward).

In some embodiments, the QGFN generation system improves generative stochastic models in building biochemical structures at each analytical step by considering both a predicted flow measure and an action-value. Specifically, the action-value estimates the predicted ultimate reward of a particular selection or structure. Moreover, because the action-value estimates ultimate reward of an action, it can be viewed as a greedy measure that focuses on high-value outcomes at the expense of exploring an action space. By combining flow metrics with an action-value, the QGFN generation system can balance space exploration with seeking high-reward outcomes. Specifically, the combination of the flow measure and the action-value can be controlled by a mixing hyperparameter (e.g., to indicate which constructive object options to mask).

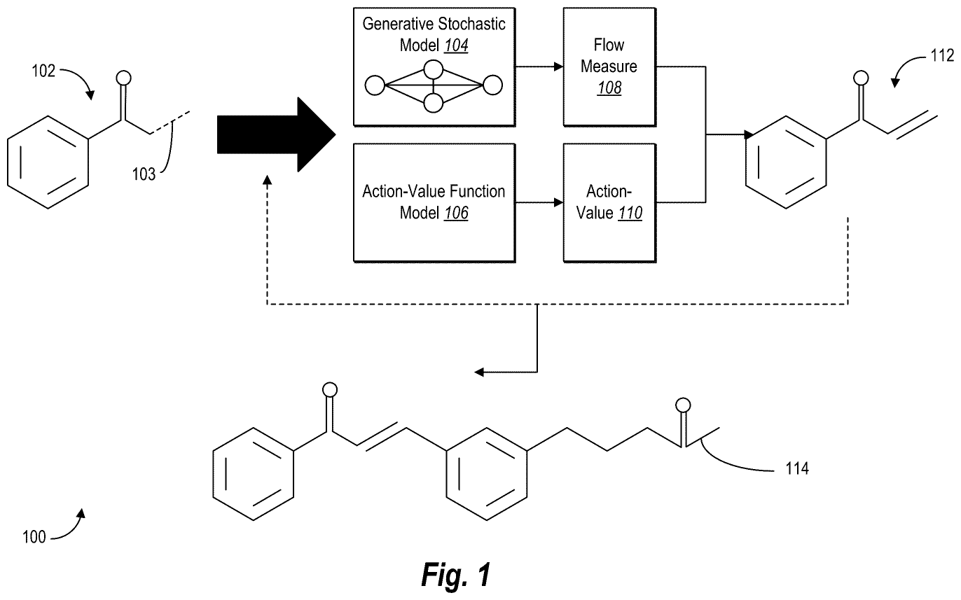

As shown, FIG. 1 illustrates an overview of a QGFN generation system 100 adding a constructive object option in generating a biochemical structure in accordance with one or more embodiments. For example, FIG. 1 illustrates the QGFN generation system 100 receiving an input state 102 of a biochemical structure. In one or more embodiments, an “input state” refers to a representation of input data. Specifically, the input state 102 can include an initial fragment (e.g., for a fragment-based molecule generation task), an input atom (for a small molecule construction task), an initial nucleobase (e.g., for an RNA-binding task). Further, the input state 102 can also include the input data after adding one or more constructive object options. In other words, the input state 102 varies according to a stage of construction of the QGFN generation system 100. Moreover, as indicated by the dotted lines for the input state 102, the QGFN generation system 100 will add a constructive object option 103 to the input state 102.

As further shown, the QGFN generation system 100 processes the input state 102 with a generative stochastic model 104 and an action-value function model 106. In one or more embodiments, a “generative stochastic model” refers to a probabilistic model that generates synthetic data or structures (e.g., from a learned statistical policy that models an environment based on observed data). Specifically, the generative stochastic model 104 analyzes an initial input state and utilizes a stochastic model to estimate a measure of flow the indicates the cumulative probability of reward for downstream paths for a particular option. For instance, a generative stochastic model can learn a stochastic policy for generating an object from a sequence of actions, such that the probability of generating an object is proportional to a reward for that object. The generative stochastic model can utilize a variety of machine learning architectures or approaches. In one or more implementations, the QGFN generation system 100 utilizes a reinforcement learning approach modeled as a flow network (e.g., utilizing temporal difference learning). For example, in one or more embodiments, the generative stochastic model can include a GFlowNet, as described in greater detail below.

In one or more embodiments, the QGFN generation system 100 utilizes the action-value function model 106 to generate a value that indicates an ultimate reward for selecting a constructive object option. Specifically, the action-value function model 106 estimates the expected highest reward from a particular input state and taking an action in that state. In contrast to the generative stochastic model 104, the action-value function model 106 estimates the ultimate or highest reward for taking an action (e.g., in contrast to the cumulative reward from available downstream paths after taking the action). For instance, an action-value function can model the probability of a policy on the highest-return sequence of actions. In other words, the QGFN generation system 100 can utilize the action-value function model 106 to prioritize greedier actions (e.g., pursue building a biochemical structure that skews towards more reward rather than diversity). An action value-function can be learned utilizing a variety of machine learning approaches, including a variety of reinforcement learning techniques. For example, in one or more implementations, the QGFN generation system 100 utilizes a Q-value function, as described in greater detail below.

As shown, the QGFN generation system 100 utilizes the generative stochastic model 104 to generate a flow measure 108 for the constructive object option 103. In one or more embodiments, the constructive object option 103 refers to an object or action that can be added to the input state 102 to build an intermediate/final biochemical structure (e.g., adding a node to a graph). To illustrate, the constructive object option 103 includes adding a fragment to a molecule, adding an atom or bond, adding a nucleobase. For example, for each stage of constructing a biochemical structure, the QGFN generation system selects a constructive object option from a plurality of constructive object options to build the biochemical structure.

In one or more embodiments, the term “flow measure” refers to a measure that indicates a cumulative probability of reward. For instance, a flow measure can be modeled as energy flow, where the energy flow is proportional to the probability of reward following from choosing a particular option. For example, the flow measure 108 indicates a total reward for selecting a constructive object option, where the reward reflects the selected constructive object option and additional downstream constructive object options.

As further shown, the QGFN generation system 100 utilizes the action-value function model 106 to generate an action-value 110 for the constructive object option 103. As mentioned, the QGFN generation system 100 utilizes the action-value function model 106 to generate an action-value 110, where the action-value 110 indicates the ultimate reward for selecting the constructive object option 103. Thus, for each constructive object option, the QGFN generation system 100 generates an action-value and a flow measure.

Accordingly, in one or more embodiments, based on a combination of the flow measure 108 and the action-value 110, the QGFN generation system 100 selects the constructive object option 103 from a plurality of constructive object options to generate an intermediate biochemical structure 112. For instance, the intermediate biochemical structure 112 refers to a partially built biochemical structure. Specifically, the intermediate biochemical structure 112 has not reached a terminal state and requires additional construction stages. As shown by a biochemical structure 114, the QGFN generation system 100 generates/builds the entire structure after multiple iterations. In other words, the QGFN generation system 100 performs multiple iterations of generating flow measures and action-values for various constructive object options until it generates the biochemical structure 114.

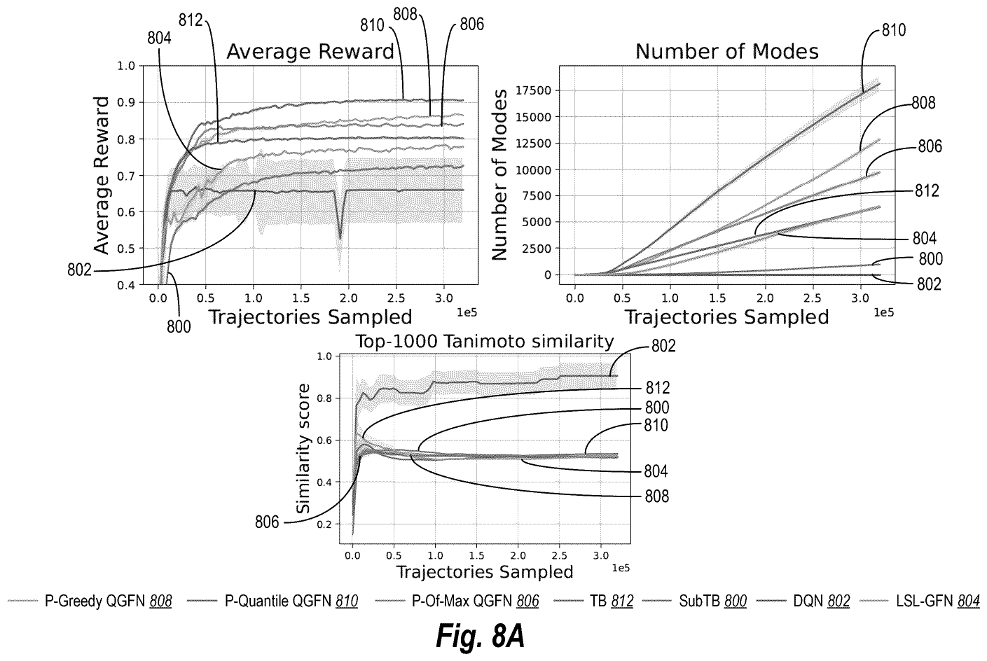

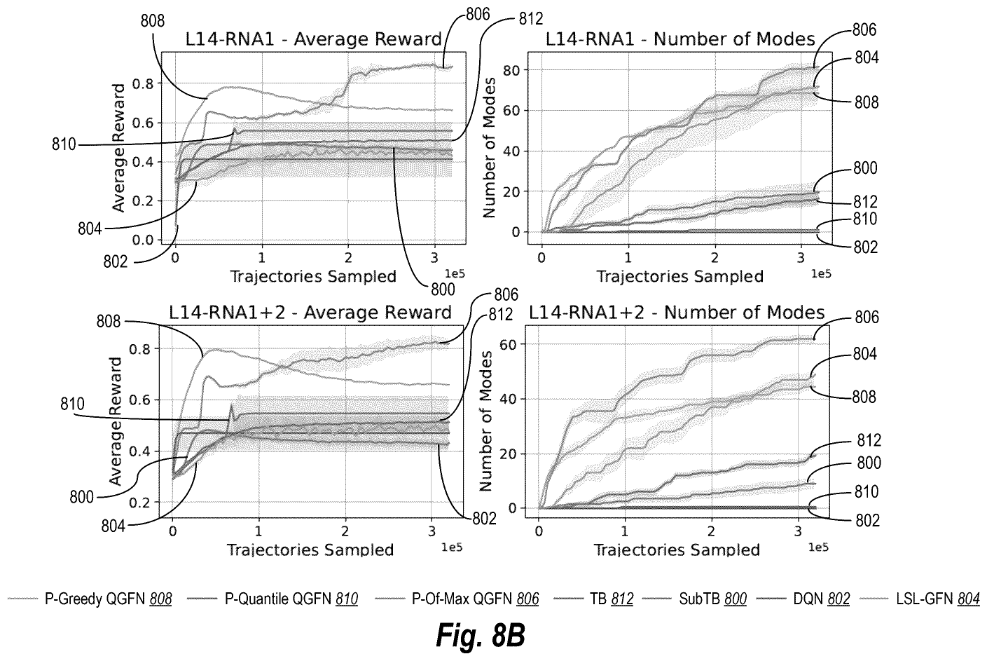

Although the description of FIG. 1 describes the QGFN generation system 100 generating biochemical structures, in one or more embodiments, the QGFN generation system 100 extends to additional spaces. Specifically, the QGFN generation system 100 can operate in a variety of complex tasks such as data processing pipelines, circuit design, machine learning pipelines, semantic parsing, and optimization problems. For instance, in some embodiments, the QGFN generation system 100 can generate a bit sequence of a specified length, which is discussed in more detail below in FIGS. 8A-8B.

As mentioned briefly above, conventional systems suffer from a number of technical deficiencies with regard to implementing computing devices. For example, conventional systems often adjust a reward parameter (e.g., beta described below) when utilizing generative methods. However, in increasing the reward parameter of generative methods (e.g., biasing the model to favor greedier approaches), conventional systems suffer from numerical instability which leads to inaccurate computations for constructing biochemical structures.

Furthermore, in some embodiments, an additional pitfall in adjusting the reward parameter when utilizing generative methods includes a collapse of space exploration. In other words, conventional systems that are tweaked to favor reward are less incentivized towards space exploration and suffer from a lack of diversity. Specifically, a collapse of space exploration leads to mode collapse and results in an inaccurate construction of biochemical structures and/or other types of structures (e.g., according to an objective in building the structure).

In addition to inaccuracy issues, conventional systems further suffer from computational inefficiencies. Specifically, conventional systems focus on utilizing generative methods which can be inconsistent with achieving an objective of constructing a biochemical structure. For instance, generative methods utilized by conventional systems typically focus on the number of options and samples many small rewards, rather than balancing space exploration with reward seeking. As a result, conventional systems inefficiently build biochemical structures when employing generative methods. Relatedly, conventional systems suffer from operational inflexibility. Specifically, conventional systems fail to flexibly balance between reward and space exploration, leading to detrimental results such as mode collapse.

In one or more embodiments, the QGFN generation system 100 overcomes the deficiencies of conventional systems. For example, in some embodiments, the QGFN generation system 100 overcomes inaccuracies of conventional systems by utilizing both a generative stochastic model and an action-value function model. Specifically, the QGFN generation system 100 generates a flow measure and an action-value for a constructive object option and combines them utilizing various approaches (as discussed below) to select a constructive object option from a plurality of constructive object options. For instance, the QGFN generation system 100 utilizing both the flow measure and the action-value allows the QGFN generation system 100 to reduce excessive bias towards reward. In other words, the QGFN generation system 100 balances reward seeking with space exploration by using a combination of the generative stochastic model and action-value function model outputs (e.g., tuned according to a hyperparameter to indicate which constructive object options to mask). As such, the QGFN generation system 100 more accurately builds biochemical structures in accordance with objectives in building the structures (e.g. an objective such as maximizing binding affinity to a specific protein, maximizing stability or reactivity, etc.).

Moreover, in some embodiments, the QGFN generation system 100 counters mode collapse, by utilizing a combination of flow measures and action-values in selecting constructive object options. As mentioned above, the generative stochastic model allows the QGFN generation system 100 to emphasize space exploration for building a biochemical structure and the action-value function model allows the QGFN generation system 100 to emphasize reward. As such, the QGFN generation system 100 combines the flow measures and action-values (e.g., to generate an action-value flow measure) to balance the focus of space exploration and reward at various steps of constructing a biochemical structure. Furthermore, in some embodiments, the QGFN generation system 100 can adjust how the flow measure and the action-value are combined at different points of constructing a biochemical structure. As such, the QGFN generation system 100 improves upon the accuracy of fulfilling objectives in building the biochemical structure by avoiding mode collapse and sampling high reward actions.

In one or more embodiments, the QGFN generation system 100 improves upon computational efficiency in building a biochemical structure by balancing accuracy and efficiency concerns. Specifically, the QGFN generation system 100 does not focus solely on space exploration (e.g., sampling many small rewards). As mentioned above, the QGFN generation system 100 balances space exploration with reward seeking in various different manners by utilizing both a generative stochastic model and an action-value function model to hone in on improved predictions without sacrificing mode diversity. In doing so, the QGFN generation system 100 improves efficiency of building a biochemical structure that conforms with various objectives in building a biochemical structure.

Related to the above, the QGFN generation system 100 improves upon operational flexibility by utilizing the generative machine learning framework that includes both the generative stochastic model and the action-value function model. For example, the QGFN generation system 100 tailors the trade-off between reward and space-exploration based on the construction task and intelligently generates the biochemical structure in a more flexible manner that better accounts for high-reward and space-exploration. Moreover, in some implementations, the QGFN generation system 100 allows for modification and variability of a combination value (e.g., p value described below) relative to training and inference. Thus, the QGFN generation system 100 can utilize various p-value hyperparameters during training and client devices can modify such p-values at inference time depending on particular contexts or applications. Moreover, the QGFN can apply different combination values utilizing different approaches at training and inference (e.g., flexibly utilize a p-greedy approach versus a p-quantile approach or another combination approach at training and/or inference).

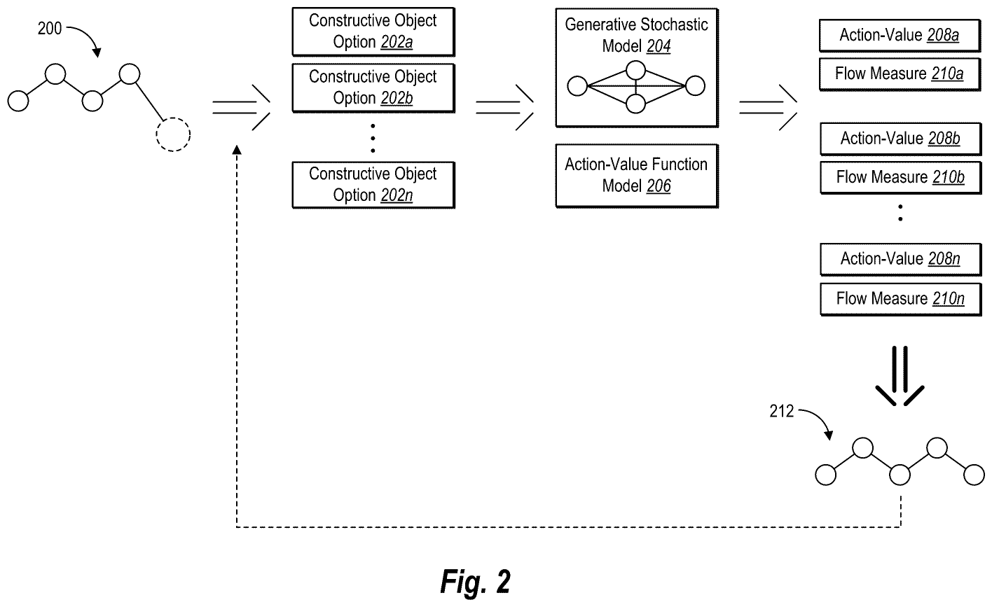

As mentioned, the QGFN generation system 100 selects a constructive object option form a plurality of constructive object options to build a biochemical structure. FIG. 2 illustrates an example diagram of the QGFN generation system 100 selecting a constructive object option based on a plurality of action-values and flow measures in accordance with one or more embodiments.

As shown, the QGFN generation system 100 receives an input state 200 of a biochemical structure. In one or more embodiments, a biochemical structure refers to an arrangement of molecules and/or atoms. Specifically, biochemical structure includes fragment-based molecules, atom-based molecules, and RNA molecules. Further, the term biochemical structure includes properties such as three-dimensional shape, topology, folding, and higher-order interactions between structures (e.g., protein complexes, nucleic acid-protein complexes, lipids, etc.).

As mentioned above, the QGFN generation system 100 builds biochemical structures in accordance with certain objectives. For example, for a fragment-based molecule generation task, the QGFN generation system 100 builds a graph of nodes that represent various molecular fragments with edges that represent the relationships between the nodes. For instance, the QGFN generation system 100 performs fragment-based molecular generation task with a reward objective tied to predicting the binding affinity of a molecule to a protein.

As a further example, for an atom-based task, the QGFN generation system 100 builds a graph of nodes that represents small molecules. For instance, the QGFN generation system 100 explores an action space that includes adding atoms or bonds with an objective of maximizing properties such as stability and/or reactivity (e.g., as a reward). Additionally, for an RNA-binding task, the QGFN generation system 100 builds a graph of nodes that represents nucleobases. For instance, the QGFN generation system 100 generates a string of nucleobases with an objective (e.g., reward) tied to maximizing the binding affinity to a target transcription factor.

As shown in FIG. 2, starting from the input state 200, the QGFN generation system 100 has a plurality of constructive object options to select from. In one or more embodiments, the QGFN generation system 100 selects a constructive object option from a plurality of constructive object options to build a biochemical structure. Specifically, the plurality of constructive object options refer to potential options for building a biochemical structure. For example, each of the plurality of constructive object options can impact the diversity (e.g., a specific mode) and reward (e.g., depending on the objective) of the overall biochemical structure.

In one or more embodiments, the QGFN generation system 100 builds a biochemical structure based on a reward of adding a particular constructive object option. Specifically, the reward refers to a value that quantifies how well a model performs for a specific task or objective. For example, an agent model makes decisions and receives feedback in the form of rewards, where the rewards indicate how desirable or undesirable an outcome of an action or a sequence of actions was. In some instances, the agent has an objective to maximize the reward. As described above, for building biochemical structures there can be a variety of objectives for a reward (e.g., prediction of a binding affinity to a specific protein, molecular properties such as stability and reactivity, predicting a binding affinity to a target transcription factor).

FIG. 2 shows a constructive object option 202a, a constructive object option 202b, and a constructive object option 202n. For each of the constructive object options 202a-202n, the QGFN generation system 100 utilizes a generative stochastic model 204. In some embodiments, the QGFN generation system 100 utilizes a generative flow network as the generative stochastic model 204. Specifically, a “generative flow network” (or “GFN”) refers to a generative framework designed to sample combinatorial objects, with diversity based on an energy function. Specifically, a generative flow network includes a reinforcement model trained with an objective of sampling a distribution of trajectories whose probability is proportional to a reward. Accordingly, a generative flow network is a machine learning approach that turns a reward into a generative policy that samples with a probability proportional to the return. For instance, a generative flow network applies flow-matching conditions where the flow incoming to a state matches the outgoing flow (proportional to the reward) which leads to learning downstream reward probabilities for any particular option. For example, in some embodiments, the QGFN generation system 100 utilizes generative flow networks in the manner described in Bengio, E., Jain, M., Korablyov, M., Precup, D., and Bengio, Y., Flow network based generative models for non-iterative diverse candidate generation, Advances in Neural Information Processing Systems, 34:27381-27394, 2021a.; Bengio, Y., Deleu, T., Hu, E. J., Lahlou, S., Tiwari, M., and Bengio, E. Gflownet foundations, arXiv preprint arXiv: 2111.09266, 2021b; and Pan, L., Zhang, D., Jain, M., Huang, L., and Bengio, Y., Stochastic generative flow networks, arXiv preprint arXiv: 2302.09465, 2023 (hereinafter “Pan”), which are fully incorporated by reference herein.

As shown, the QGFN generation system 100 utilizes the generative stochastic model 204 to generate a plurality of flow measures 210a-210n. As discussed above, the plurality of flow measures 210a-210n emphasize diverse modes over greedier actions.

Further, as shown, the QGFN generation system 100 utilizes an action-value function model 206 to generate a plurality of action-values 208a-208n for each of the plurality of constructive object options 202a-202n. As mentioned above, the QGFN generation system 100 utilizes the action-value function to generate the action-values that indicate ultimate rewards for selecting a particular constructive object option, rather than cumulative downstream rewards. For instance, the action-value function model 206 can include a learned model that estimates the highest ultimate reward from a particular action. In other words, the generated action-values 208a-208n emphasize greedier actions instead of diversity of modes. For example, in one or more implementations, the QGFN generation system 100 utilizes action-value functions in a manner described in Sutton, R. S. and Barto, A. G., Reinforcement learning: An introduction. MIT press, 2018; Watkins, C. J. and Dayan, P., Q-learning, Machine learning, 8:279-292, 1992; and Mnih, V., Kavukcuoglu, K., Silver, D., Graves, A., Antonoglou, I., Wierstra, D., and Riedmiller, M., Playing atari with deep reinforcement learning, arXiv preprint arXiv: 1312.5602, 2013, which are fully incorporated by reference herein.

As shown, the QGFN generation system 100 selects a constructive object option from the plurality of constructive object options to add to the input state 200. Specifically, the QGFN generation system 100 selects the constructive object option based on the plurality of flow measures 210a-210n and the plurality of action-values 208a-208n to generate an intermediate biochemical structure 212. Moreover, as indicated by the dotted arrows from the intermediate biochemical structure 212, the QGFN generation system 100 can perform additional iterations for further constructing the biochemical structure from the intermediate biochemical structure 212 as an input state.

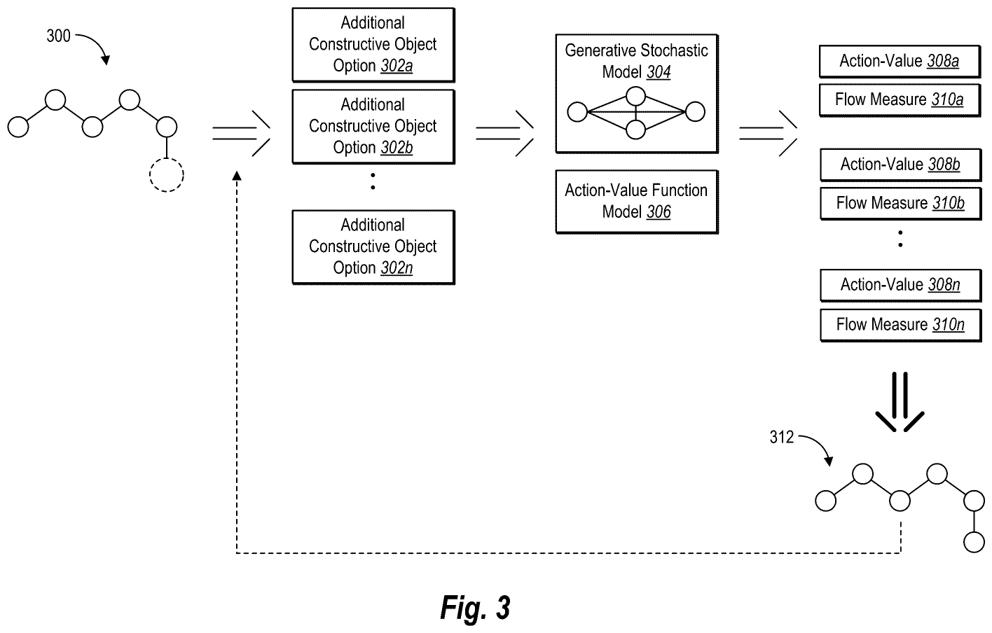

As mentioned above, the QGFN generation system 100 can build a biochemical structure with multiple construction stages. FIG. 3 illustrates an example diagram of the QGFN generation system 100 adding an additional constructive object option to an intermediate biochemical structure in accordance with one or more embodiments. For instance, FIG. 2 illustrated a first construction stage for the intermediate biochemical structure, while FIG. 3 illustrates a second construction stage.

As shown, the QGFN generation system 100 processes an input state 300 (e.g., the intermediate biochemical structure 212 described above in FIG. 2). As mentioned above, the QGFN generation system 100 takes an input state 300 and builds a graph to represent the input state 300. For instance, the QGFN generation system 100 builds a graph to represent a particular instantiation or configuration of the intermediate biochemical structure 212.

As mentioned, FIG. 3 illustrates a second construction stage of adding a constructive object option to the input state 300. Specifically, as shown, the QGFN generation system has an additional plurality of constructive object options 302a-302n. Similar to FIG. 2, as shown here in FIG. 3, the QGFN generation system 100 utilizes a generative stochastic model 304 and an action-value function model 306 to generate a plurality of additional action-values 308a-308n and a plurality of additional flow measures 310a-310n. For instance, the action-value 308a and the flow measure 310a correspond to the additional constructive object option 302a.

As shown, for the second construction stage, the QGFN generation system 100 selects from the additional plurality of constructive object options 302a-302n to add to the intermediate biochemical structure 312. Specifically, the QGFN generation system 100 selects from the additional plurality of constructive object options 302a-302n based on the plurality of additional action-values 308a-308n and the plurality of additional flow measures 310a-310n. In doing so, the QGFN generation system 100 generates an additional intermediate biochemical structure 312. For instance, as indicated by the dotted arrows from the additional intermediate biochemical structure 312, the QGFN generation system 100 continually iterates additional construction stages until a termination state is reached (e.g., a biochemical structure is fully built).

As mentioned above, in some embodiments, the QGFN generation system 100 utilizes a generative flow network as the generative stochastic model 304. For example, as mentioned, the QGFN generation system 100 creates a state space described by a DAG where G=(S, A), and where s∈S is a partially constructed object, and (s→s′)∈A⊂S×S is a valid additive step.

In some embodiments, the QGFN generation system 100 optimizes generative flow networks to satisfy balance conditions of flow. As discussed above, flow measures indicate a total cumulative reward. For further elaboration, QGFN generation system 100 models the flow measures (F(s)) such that the flows going through states are conserved (e.g., an input state such as an intermediate biochemical structure). Specifically, terminal states (e.g., corresponding to fully constructed biochemical structures) absorb non-negative units of flow, and intermediate states have as much flow coming into them (from parent nodes) as flow coming out of them (to children nodes). To illustrate, in some embodiments, the QGFN generation system 100 represents flow measures for a partial trajectory (sₙ, . . . , sₘ) (e.g., incomplete trajectories that have not reached a fully constructed biochemical state) as follows:

$$F ( {s}_{n} ) {\prod }_{i = n}^{m - 1} {P}_{F} ( {s}_{i + 1} \vert {s}_{i} ) = F ( {s}_{m} ) {\prod }_{i = n}^{m - 1} {P}_{B} ( {s}_{i} \vert {s}_{i + 1} )$$

In the above notation, P_{F} and P_{B} represent forward and backward policies, respectively. Specifically, the forward policy and the backward policy represents distributions over children and parents of flow emanating forward and backward from a specific state. For instance, the QGFN generation system 100 constructs for terminal (leaf) states as follows F(s)=R(s). Another way that the QGFN generation system 100 represents the forward backward policies is through edge flows as follows F(s→s′)=F(s)P_{F} (s′|s). Moreover, in some embodiments, the QGFN generation system 100 represents flow conditions as preserving incoming flows and outgoing flows for all states s∈S as:

$${\sum }_{{s}_{i} ∈ aPar ( s )} F ( {s}_{i} \to s ) = {\sum }_{{s}_{o} ∈ Chld ( s )} F ( s \to {s}_{o} )$$

By constructing the edge flow F(s→S_{T}) to a terminal state S_{T}, the QGFN generation system 100 represents this as R(S_{T}) which indicates the flow corresponding to taking a stop action, and the initial state Sₒ which has no parents, only has to account for the flow of its children (e.g., because it is a source in the network).

In one or more embodiments, the QGFN generation system 100 utilizes learning objectives such as trajectory balance (e.g., where n=0 and m is the trajectory length) and sub-trajectory balance (e.g., where all combinations of (n, m) are used). For instance, the QGFN generation system 100 utilizes trajectory balance in the manner described in Malkin, N., Jain, M., Bengio, E., Sun, C., and Bengio, Y. Trajectory balance: Improved credit assignment in gflownets, Advances in Neural Information Processing Systems, 35:5955-5967, 2022a, which is fully incorporated by reference herein. Further, the QGFN generation system 100 utilizes sub-trajectory balance as described in Madan, K., Rector-Brooks, J., Korablyov, M., Bengio, E., Jain, M., Nica, A. C., Bosc, T., Bengio, Y., and Malkin, N, Learning gflownets from partial episodes for improved convergence and stability, In International Conference on Machine Learning, pp. 23467-23483. PMLR, 2023, which is fully incorporated by reference herein.

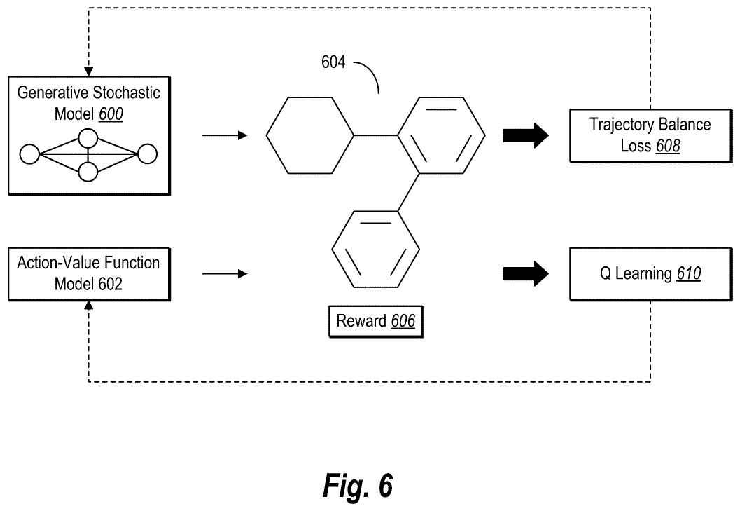

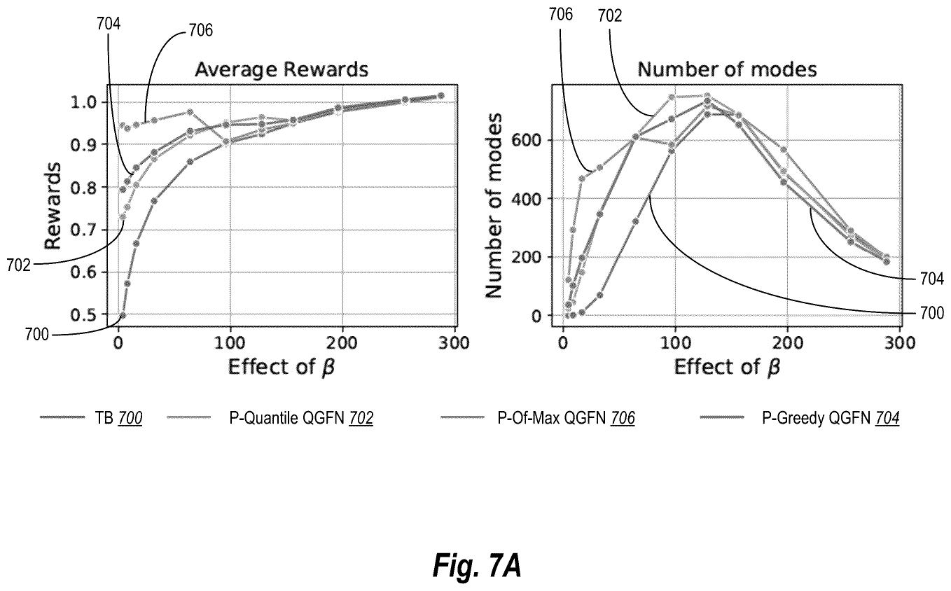

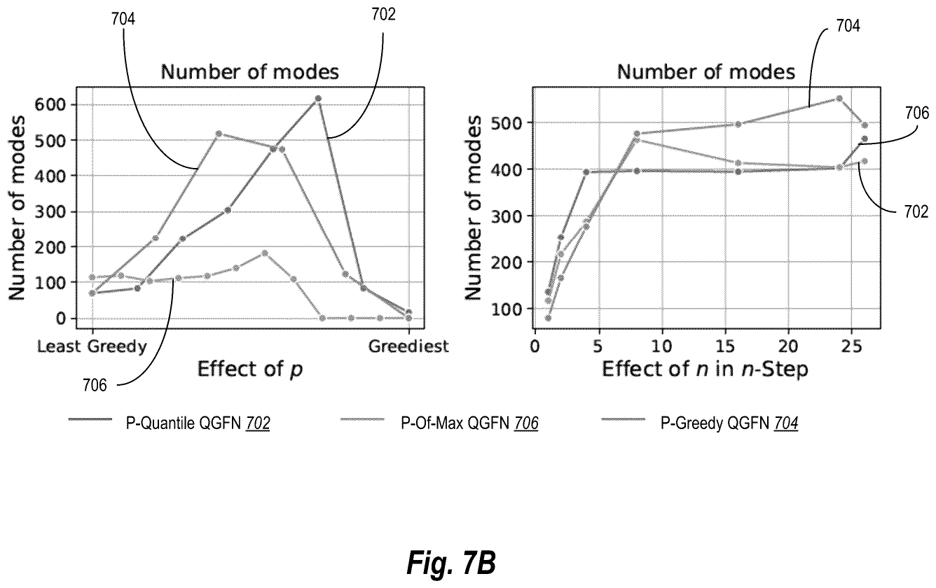

By satisfying the conditions of trajectory balance or sub trajectory balance (e.g., 0 loss everywhere), the QGFN generation system 100 samples terminal states with a probability proportional to the reward of the completing the biochemical structure. Moreover, during construction of (e.g., generation) the biochemical structure, the relationship between the flow (F) and the forward policy (P_{F}) is such that, if s→s′∈A, then P_{F}(s′|s)=P_{B}(s|s′)F(s′)/F(s)αF(s′). In other words, the likelihood of going from s to s′ is proportional to the flow in s′. Additional details of training the generative stochastic model utilizing trajectory balance loss is given below in the description of FIG. 6. Moreover in FIGS. 7A-8B, experimental results relating to trajectory balance and sub trajectory balance are described as the generative flow network objectives (e.g., baselines to compare performance of the QGFN generation system 100 with other systems).

Moreover, in reinforcement learning, the action-value function Q^{π}(s, a) estimates the expected reward-to-go. For Q^{π} there are several possible policy choices, thus Q^{π} can be referred to as Q when statements apply to a large number of policies.

As mentioned above, the action-value indicates the expected (e.g., ultimate) reward when following a policy π starting in some state s and taking action a (e.g., for a discount factor [symbol] that represents the importance of future rewards relative to immediate rewards, where in reinforcement learning the importance of future rewards is discounted by a factor of [symbol] at each step). In some embodiments, the QGFN generation system 100 utilizes a discount factor of 1 to avoid arbitrarily penalizing larger biochemical structures (e.g., structures that involve many construction stages). For instance, the QGFN generation system 100 represents the action-value function model as follows:

$${Q}^{π} ( s , a ) = {}_{{s}_{t + 1} ∼ T ( {s}_{t} , {a}_{t} )}^{𝔼} {a}_{t}^{} ∼ π ( \cdot \vert {s}_{t} ) [ {\sum }_{t = 0}^{∞} {γ}^{t} R ( {s}_{t} ) \vert {s}_{0} = s , {a}_{0} = a ]$$

In the above notation, T(s, a) represents a stochastic transition operator (e.g., a description of the dynamics of an environment that specifies the probabilities of transitioning from one state to another based on the actions taken by an agent, in other words it introduces randomness or uncertainty by describing the probability distribution over possible next states given the current state and action).

As applied to the QGFN generation system 100, the stochastic transition operator in a generative flow network context includes constructing objects in a deterministic setting, which would include stochastic extensions (e.g., stochastic transition operators that introduce randomness into fixed/deterministic systems).

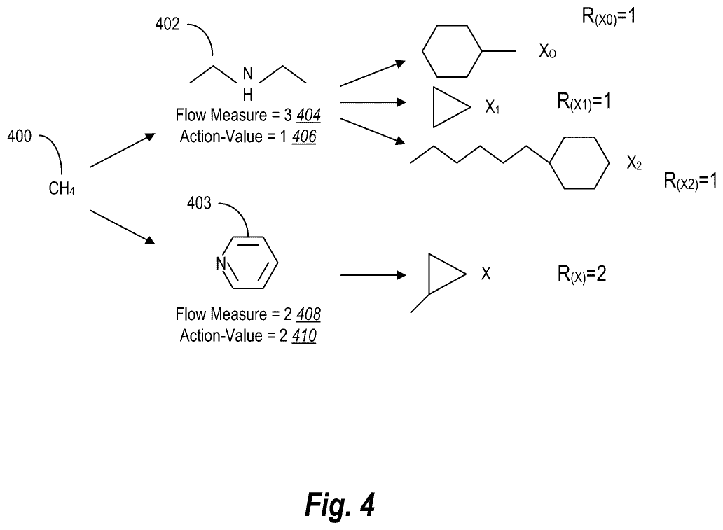

As mentioned above, the action-value differs from the flow measure and the QGFN generation system 100 utilizes both to balance greedy actions with exploring an action space. FIG. 4 illustrates an example diagram of the difference between a flow measure and an action-value in accordance with one or more embodiments. For example, FIG. 4 shows an input state 400 and potential constructive object options to add to the input state 400.

FIG. 4 provides an illustration of the input state 400 (CH₄) with the option to select from a first constructive object option 402 and a second constructive object option 403. Further, FIG. 4 illustrates that downstream from the first constructive object option 402 are additional constructive object options (e.g., actions to take after selecting the first constructive object option 402). For instance, FIG. 4 shows X₀, X₁, and X₂, each with a reward of 1 (e.g., R₍ₓ₎=1). Further, FIG. 4 shows that downstream from the second constructive object option 403 is an additional constructive object option (X). As shown, the additional constructive object option (X), has a reward R₍ₓ₎ of 2.

As illustrated, because the first constructive object option 402 contains three downstream options, a flow measure 404 for the first constructive object option 402 is three. Whereas the second constructive object option 403 contains one downstream option, such that a flow measure 408 is two (e.g., equaling the reward of the single downstream option). In such an instance, focusing on the flow-measures alone can result in the QGFN generation system 100 selecting the first constructive object option 402, due to the greater flow measure 404. However, as illustrated, an action-value 406 for the first constructive object option 402 is one and an action-value 410 for the second constructive object option 403 is two. As such, considering the action-values allows for the QGFN generation system 100 to prioritize greedier actions.

To reiterate, the QGFN generation system 100 utilizes a combination of the flow measure and the action-value, especially in situations with a very large search space. To illustrate, a molecular design task can have reward that ranges from [0, 1]. Further, there can be 10⁶ molecules with a reward of 9 but just a dozen with a reward of 1. Since 0.9×10⁶ is much greater than 12×1, the probability of sampling a reward 1 molecule will be low if naively using this reward. Rather than merely just adjusting beta (e.g., a temperature parameter for generative stochastic networks that focus on greedy actions but can lead to mode collapse), the QGFN generation system 100 implements the complementary combination of a generative flow network and an action-value function model (e.g., which can be further adjusted at inference time to focus on greediness or space exploration).

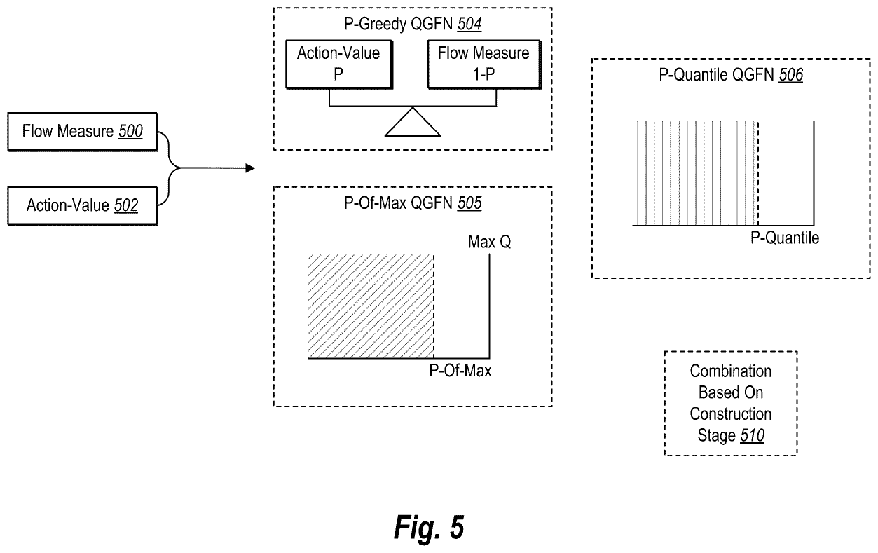

As mentioned above, the QGFN generation system 100 combines the action-value and the flow measure in a variety of ways. FIG. 5 illustrates an example diagram of the QGFN generation system 100 utilizing the model outputs to select a constructive object option from a plurality of constructive object options in accordance with one or more embodiments.

As shown, the QGFN generation system 100 generates a flow measure 500 and an action-value 502 and combines/utilizes both metrics. In some embodiments, the QGFN generation system 100 utilizes both metrics via p-greedy QGFN 504. Specifically, the QGFN generation system 100 utilizes p-greedy QGFN 504 to balance a cumulative reward indicated by the flow measure and an ultimate reward indicated by the action-value.

For example, p-greedy QGFN 504 includes defining a behavior policy to include a mixture between a forward policy and a greedy policy with a factor of p. Specifically, the p-greedy QGFN 504 includes the QGFN generation system 100 generating an action-value flow measure (e.g., a combination of the flow measure and the action-value). Moreover, the p-greedy QGFN 504 includes the QGFN generation system 100 balancing the flow measure with the action-value of a constructive object option according to a combination value (e.g., the combination value is a factor of p, in other words, the combination value is a mixing hyperparameter that indicates how to mix the flow measure and the action-value). For instance, the QGFN generation system 100 represents the p-greedy QGFN 504 as:

$$μ ( {s}^{′} \vert s ) = ( 1 - p ) {P}_{F} ( {s}^{′} \vert s ) + p [ {s}^{′} = arg {max}_{i} Q ( s , i ) ]$$

The above notation indicates that the QGFN generation system 100 follows the forward policy (P_{F}) but picks the greedy action according to the action-value (Q) with probability p. For instance, the above notation indicates taking 1 minus the combination value (p) multiplied by the flow measure and combining that with the combination value (p) multiplied by the action-value) to arrive at the behavioral policy (u).

In some embodiments, the QGFN generation system 100 utilizes p-greedy QGFN 504 to sample from an original generative flow network to choose an action-value (Q) with probability p. In other words, lowering the probability p, results in the QGFN generation system 100 taking greedier actions (e.g., a high combination value would indicate a balance in favor of the action-value while a low combination value would indicate a balance in favor of the flow measure). To illustrate, for a p-value of 0.5, half of the constructive object options are selected according to the generative flow network (e.g., the flow measures), and half are selected according to the action-value function model (e.g., the action-values).

As further shown, in some embodiments, the QGFN generation system 100 utilizes a p-of-max QGFN 505. Specifically, the p-of-max QGFN 505 includes determining a plurality of flow measures and a plurality of action-values corresponding to a plurality of constructive object options and selecting an action-value threshold based on the plurality of action-values. For example, the p-of-max QGFN 505 includes defining a behavior policy (μ) as a masked version of the forward policy P_{F} (e.g., masking certain flow measures that fail to satisfy an action-value threshold). For instance, constructive object options with action-values (e.g., Q-values) less than p maxₐ Q(s, a) have a probability of 0. In other words:

$$μ ( {s}^{′} \vert s ) α {P}_{F} ( {s}^{′} \vert s ) [ Q ( s , {s}^{′} ) \geq \begin{matrix}max \\ i\end{matrix} Q ( s , i ) ]$$

The above notation indicates that the max possible action-value (Q) is multiplied by the p-value, to establish the action-value threshold. In other words, the QGFN generation system 100 generates an action-value threshold by combining the combination value (p) with a max action-value (Q) from a plurality of constructive object options. All values below the action-value threshold are masked. To illustrate, for a p-value of 0.9, and for a max action-value (Q) of 1, all constructive object options with a flow measure below 0.9 are masked.

In one or more embodiments, the QGFN generation system 100 utilizes p-quantile QGFN 506. For example, utilizing the p-quantile QGFN 506 includes masking one or more constructive object options of the plurality of constructive object options by applying an action-value threshold to action-values corresponding to the one or more constructive object options. Further, p-quantile QGFN 506 includes selecting the constructive object option from the plurality of constructive object options based on the flow measure and the constructive object option not being masked. Specifically, the p-quantile QGFN 506 includes defining a behavioral policy as a masked version of the forward policy, where actions below a p-quantile (e.g., a changeable p quantile) of the action-value have a probability of 0 (e.g., they are masked). For instance, let qₚ(Q, s) be p-quantile of Q(s,⋅), then:

$$μ ( {s}^{′} \vert s ) α {P}_{F} ( {s}^{′} \vert s ) [ Q ( s , {s}^{′} ) \geq {q}_{p} ( Q , s ) ]$$

In other words, p-quantile QGFN 506 includes following the forward policy but discarding actions whose value is in the bottom p % of action-values (Q). To illustrate, the QGFN generation system 100 utilizes p-quantile QGFN 506 by taking all the action-values (Q), sorting them, and obtaining the p quantile (e.g., by determining the 75^{th} percentile of action-values). For example, every constructive object option below the p quantile is masked (e.g., any path below the p quantile is closed down, because the action-value for that path indicates a low reward). In some embodiments, the p-quantile QGFN 506 more aggressively prioritizes greedy actions by pruning the search space to remove constructive object options which the action-value estimates as not leading to high reward outcomes.

In some embodiments, the QGFN generation system 100 utilizes a combination based on a construction stage 510. For instance, the QGFN generation system 100 determines a construction stage threshold that indicates a number of construction stages before switching methods. For instance, the QGFN generation system 100 establishes a construction stage threshold of utilizing a first method for the first half of construction (e.g., 5 steps) and utilizing a second method for the second half of construction (e.g., the latter 5 steps).

To illustrate, for building a molecule (e.g., fragment-based molecular generation), the QGFN generation system 100 can utilize p-quantile QGFN 506 to prioritize pruning of constructive object options down to ones with higher reward. As the construction for a molecule progresses, the QGFN generation system 100 can switch to p-greedy QGFN 504 to prioritize space exploration.

To further illustrate, for RNA sequencing there are often much fewer constructive object options than molecular generation. As such, in one or more implementations, the QGFN generation system 100 avoids utilizing p-quantile QGFN 506 and utilizes p-of-max QGFN 505 and/or p-greedy QGFN 504. In other words, the QGFN generation system 100 utilizes a combination of different methods depending on a stage of construction and the objectives at each stage.

In one or more embodiments, the QGFN generation system 100 can utilize a combination value that prioritizes greedier actions (e.g., Q) for a predetermined number of initial steps for construction. Further, the QGFN generation system 100 then transitions from prioritizing greedier actions (e.g., Q) to prioritizing space exploration (e.g. P_{F}) for the remaining construction steps. In other words, the QGFN generation system 100 changes the combination value (p) as the construction stages progress. For instance, the QGFN generation system 100 starts with a higher combination value (p) to prioritize greedier actions and at later stages utilizes a lower combination value (e.g., which could prioritize space exploration), or vice versa. In some embodiments, the QGFN generation system 100 switches methods at each construction stage.

As mentioned above, the QGFN generation system 100 trains the generative stochastic model and the action-value function model. FIG. 6 illustrates the QGFN generation system 100 jointly training a generative stochastic model and an action-value function model in accordance with one or more embodiments. For example, the QGFN generation system 100 trains a generative stochastic model 600 and an action-value function model 602 online (e.g. sampling data from some behavior policy u and training the flow (F), the forward policy P_{F}, and the backward policy P_{B} to minimize a flow consistency loss on the sampled data).

As shown in FIG. 6, the QGFN generation system 100 generates a completed biochemical structure 604 with a corresponding reward 606. For instance, the QGFN generation system 100 trains the generative stochastic model 600 and the action-value function model 602 on a variety of behavior policies and combines the action-value and the flow measure to form a greedier behavior policy that is modulated by a p-value (e.g., that ranges from [0, 1]).

In some embodiments, the QGFN generation system 100 generates the corresponding reward 606 by utilizing a variety of the methods described in FIG. 5 with varying p values. For instance, during training, the QGFN generation system 100 utilizes a low p-value (e.g., 0.4) for the p-quantile QGFN and p-greedy QGFN. In some instances, the QGFN generation system 100 varies the p-value between 0 and 1 for p-quantile QGFN and p-greedy QGFN. Moreover, in some instances during training, the QGFN generation system 100 utilizes a p-value between 0.9 and 1 for p-of-max QGFN to obtain the reward 606.

As further indicated, from the completed biochemical structure 604 with the corresponding reward 606, the QGFN generation system 100 further determines a trajectory balance loss 608 and Q learning 610 to modify parameters of the models. Specifically, the QGFN generation system 100 modifies parameters of the generative stochastic model 600 with the trajectory balance loss 608 and the action-value function model 602 with the Q learning 610.

As mentioned previously, the QGFN generation system 100 utilizes trajectory balance to minimize the flow consistency loss. For example, the QGFN generation system 100 utilizes the trajectory balance to train a model such that the probability of a trajectory (e.g., building a completed biochemical structure) is proportional to the reward obtained upon completion of the biochemical structure. Specifically, the trajectory balance acts as an objective for the generative stochastic model 600, where the trajectory balance loss 608 contains a relationship of the product of all the forward policy flows divided by the reward times the product of all the backward policy flows. For a more thorough treatment of the trajectory balance loss 608, see Bengio, Y., Deleu, T., Hu, E. J., Lahlou, S., Tiwari, M., and Bengio, E., Gflownet foundations, arXiv preprint arXiv: 2111.09266, 2021b, which is fully incorporated by reference herein.

In one or more embodiments, the QGFN generation system 100 determines the Q learning 610 from the completed biochemical structure 604 and the corresponding reward 606, where the Q learning 610 is a subset of reinforcement learning and indicates an update to action-values generated during constructing the completed biochemical structure 604. Accordingly, the QGFN generation system 100 updates the action-value function model 602 to reflect the corresponding reward 606 obtained upon completion of the biochemical structure 604. For example, the QGFN generation system 100 utilizes a sub-set of reinforcement learning that gives a reward upon completion of the biochemical structure 604 (e.g., in some embodiments the QGFN generation system utilizes Q-learning and/or step Q-learning to train the action-value function model 602).

As mentioned above, the QGFN generation system 100 extends to a variety of spaces, such as fragment-based molecule generation tasks, atom base tasks (QM9), RNA-binding task, and prepend-append bit sequences. To illustrate, for a fragment-based molecule generation task, the QGFN generation system 100 generates a completed fragment-based molecule structure with a corresponding reward. For instance, the QGFN generation system 100 trains the generative stochastic model 600 and the action-value function model 602 on behavior policies related to the fragment-based molecule structure (e.g., binding affinity of the molecule to a specific protein) and combines the action-value and the flow measure to form a greedier behavior policy that is modulated by a p-value (e.g., that ranges from [0, 1]). Accordingly, during implementation, the QGFN generation system 100 generates a graph of up to a specified fragments where the reward is based on an objective (e.g., binding affinity) to select constructive object options and build a completed molecule structure.

In one or more embodiments, the QGFN generation system 100 generates small molecules with a specified number of atoms. Specifically, the QGFN generation system 100 generates small molecules by parts using predefined building blocks that includes a sequence of additive edits (e.g., given a molecule and constraints of chemical validity, the QGFN generation system 100 selects an atom to attach a block to). In other words, the action space for small molecule construction is a product of determining where to attach a block and choosing which type of block to attach. Moreover, the reward for small molecule generation includes a binding energy of a molecule to a particular target (e.g., a protein target).

For instance, the QGFN generation system 100 utilizes the methods described in Bengio, E., Jain, M., Korablyov, M., Precup, D., and Bengio, Y., Flow network based generative models for non-iterative diverse candidate generation, Advances in Neural Information Processing Systems, 34:27381-27394, 2021a, for performing fragment-based molecule generation tasks and atom based tasks (which is incorporated by reference above).

To further illustrate, in some embodiments, the QGFN generation system performs RNA-binding tasks. Specifically, the QGFN generation system 100 has a smaller number of constructive object options to select from in the action space, because RNA-binding tasks involves four tokens: adenine (A), cytosine (C), guanine (G), and uracil (U). For instance, the QGFN generation system 100 trains the generative stochastic model 600 and the action-value function model 602 on behavior policies related to predicted binding affinity to a target transcription factor and combines the action-value and the flow measure to form a greedier behavior policy that is modulated by a p-value (e.g., that ranges from [0, 1]). For example, the QGFN generation system 100 utilizes the methods described in Lorenz, R., Bernhart, S. H., Honer zu Siederdissen, C., Tafer, H., Flamm, C., Stadler, P. F., and Hofacker, I. L., ViennaRNA package 2.0, Algorithms for molecular biology, 6:1-14, 2011, which is fully incorporated by reference herein.

Also, the QGFN generation system 100 utilizes baselines such as trajectory balance, sub-trajectory balance, and LSL-GFN (e.g., learning to scale logits, which is another method to control greediness), which are described in Kim, M., Ko, J., Zhang, D., Pan, L., Yun, T., Kim, W., Park, J., and Bengio, Y., Learning to scale logits for temperature-conditional gflownets, arXiv preprint arXiv: 2310.02823, 2023.

As mentioned above, the QGFN can balance high reward with space exploration, where space exploration includes selecting a diverse set of constructive object option modes. In some embodiments, a mode refers to a high-reward object that is separated from previous modes by some distance threshold. The distance function and threshold utilized depends on the task, as well as a minimum reward threshold for an object to be considered a mode.

Across various tasks, the QGFN generation system 100 demonstrates superior results in terms of higher rewards and finding a higher number of modes (e.g., high-reward, dissimilar biochemical structures or other completed objects such as bit sequences). To reiterate, the QGFN generation system 100 leverages the strengths of the generative stochastic model 600 (e.g., to cover the state space) and the action-value function model 602 (e.g., to model the expected reward of a particular action) which guides the QGFN generation system 100 to select high-expected reward branches. In other words, the QGFN generation system 100 utilizes the generative stochastic model 600 to estimate how many high-reward objects are in different parts of the state space (so that the QGFN generation system 100 goes to all important region of the state space) and further utilizes the action-value function model 602 to emphasize reward through the generated action-values to find objects with reward past the mode threshold.

Although FIG. 6 shows the QGFN generation system 100 jointly training the generative stochastic model 600 and the action-value function model 602 with the same learning rate, in some embodiments, the QGFN generation system 100 separately trains the generative stochastic model 600 and the action-value function model 602. In some embodiments, the QGFN generation system 100 can use a low p-value to train the generative stochastic model 600 (to avoid mode collapse) and train the action-value function model 602 with a higher p-value.

Moreover, although the above description describes the p-value as a selected value that can vary with different training iterations, in some embodiments, the QGFN generation system 100 learns the p value for a specific state space. Specifically, the QGFN generation system 100 identifies the optimal p value for a particular action space. For example, the QGFN generation system 100 determines a threshold for unique modes for a specific state space and identifies a satisfactory trade-off between the average reward and the average diversity. For instance, over time, the QGFN generation system can identify the optimal p value that optimizes for a number of unique modes generated by combining the generative stochastic model 600 and the action-value function model 602.

FIGS. 7A-7B illustrate the effect of training with different parameters (e.g., modifying conventional parameters relative to experimental examples of the QGFN generation system 100). FIG. 7A shows the impact of modifying the beta value (e.g., a greedy parameter for the generative stochastic model), resulting in significant mode reduction. For example, FIGS. 7A-7B illustrate the average reward and number of modes when taking 1000 samples after training is done in the fragment task (e.g., a fragment-based molecular generation task as described above in FIG. 6). As shown in FIG. 7A, increasing beta increases the average reward of the agent (e.g., the QGFN generation system 100). However, as also illustrated by FIG. 7A (e.g., the graph on the right), at some point the increase in beta causes a collapse in diversity (e.g., for TB 700 (trajectory balance), p-quantile QGFN 702, p-of-max QGFN 706, and p-greedy QGFN 704. In other words, FIG. 7A shows that too high of a beta results in a collapse of diversity around high-reward points and an inability for the model to further explore. To emphasize, the QGFN generation system 100 does not require drastic adjustment of beta to a high beta value to obtain a high average reward.

FIG. 7B illustrates the effect of modifying n (e.g., the number of bootstrapping steps in reinforcement learning for the action-value function model) and p (e.g., the combination value, or in other words, a mixture parameter for the action-value and the flow measure) during training. FIG. 7B shows that changing the p value can control greediness without necessarily causing a collapse. For instance, FIG. 7B (the graph on the left) shows that for the p-quantile QGFN 702, the p-of-max QGFN 706, and the p-greedy QGFN 704, the increase of p for a greedier effect does not necessarily lead to a collapse in the number of modes. Specifically, for the p-quantile QGFN, the number of modes peaks at a high p value.

As indicated by the graph on the right for FIG. 7B, increasing n is generally beneficial. In other words, training the action-value function model with 1-step returns is ineffective and produces poor and non-useful approximations of the action value. However, FIG. 7B (graph on the right) indicates that the increasing n (n=8 or n equaling the maximum length of a trajectory) start to show a high number of modes.

FIG. 8A illustrates experimental results for a fragment-based molecule generation task described above in FIG. 6. Specifically, FIG. 8A shows (on the top left graph) the average rewards over training trajectories. For example, FIG. 8A (top left graph) illustrates that for a greater number of training trajectories, there is typically a greater average reward for p-greedy QGFN 808, p-quantile QGFN 810, p-of-max QGFN 806, trajectory balance 812, sub trajectory balance 800, DQN 802 (e.g., deep Q-network, which is a type of reinforcement learning algorithm that combines Q-learning with deep neural networks), and LSL-GFN 804. Moreover, FIG. 8A (top right graph) further shows that the number of unique modes with a reward threshold exceeding 0.90 and pairwise similarity scores (e.g., Tanimoto, which is also known as a Jaccard index and indicates a measure of similarity between two sets) of less than 0.70. Additionally, FIG. 8A (bottom middle graph) also shows the average pairwise similarity score (Tanimoto) for the top 1000 molecules sampled by reward.

To illustrate, FIG. 8A shows that for molecular generation tasks, the p-quantile QGFN 810, returns the highest average reward as the number of trajectories sampled increases. Moreover, p-greedy QGFN 808 comes next in terms of highest average reward per trajectories sampled followed by p-of-max QGFN 806. Likewise, the number of modes follows a similar trend. Thus, FIG. 8A illustrates the best methods (discussed in FIG. 5) for combining the action-value and the flow measure that involve a large complex exploration space (e.g., a fragment-based molecular generation task).

FIG. 8B illustrates experimental results for RNA-binding tasks described above in FIG. 6. Specifically, FIG. 8B (top left graph) shows average reward and modes for an L14RNA1 task (e.g., a first binding target). Moreover, FIG. 8B (top right graph) shows the corresponding number of modes for the trajectories sampled. Additionally, FIG. 8B (bottom left) shows for L14-RNA1+2 (e.g., a second binding target), the average reward per trajectories sampled and the bottom right graph shows the number of modes for the trajectories sampled.

To illustrate, FIG. 8B shows that for RNA generation tasks, p-of-max QGFN 806 yields higher average rewards as the trajectories sampled increases, followed by p-greedy QGFN 808, and followed by p-quantile QGFN 810 (e.g., the converse of the molecular generation task). Likewise, the number of modes follows a similar trend, however, the p-quantile QGFN 810 performs more poorly than some of the other methods shown in FIG. 8B. Thus, as mentioned above, FIG. 8B illustrates the best methods (discussed in FIG. 5) for combining the action-value and the flow measure that involve a smaller complex exploration space, such as RNA generation tasks.

In addition to the illustrations in FIGS. 8A-8B, the following description illustrates an example of the QGFN generation system 100 operating in a non-biochemical space. For example, the QGFN generation system 100 can generate a bit sequence of length of 120 in a prepend-append MDP utilizing the principles described above and below (e.g., where prepend refers to adding an element to the beginning of an array, append refers to adding an element to the end of an array, and where MDP refers to a Markov Decision Process used for modeling decision-making situations). In a bit sequence generation task, |X| is limited to {0,1}ⁿ and has a state space of 2¹²⁰≈10³⁶. For a sequence of length n, R(x)=exp(1−min_{y∈M}d(x, y)/n), a sequence is considered as a unique mode if it is within the edit distance threshold 8 from M. In other words, the objective reward in the prepend-append MDP task involves constructing a bit sequence a certain distance away from M, where M is described in Malkin, N., Jain, M., Bengio, E., Sun, C., and Bengio, Y., Trajectory balance: Improved credit assignment in gflownets, Advances in Neural Information Processing Systems, 35:5955-5967, 2022a. Thus, the QGFN generation system 100 operates in this prepend-append MDP task utilizing the principles discussed above.

In some embodiments, the QGFN generation system 100 trains the models (GFN and Q) at a different p value than the p value used at inference time. As mentioned above, the QGFN generation system 100 utilizes parameters of a model trained with p-greedy QGFN with a p-value of 0.4. With this model, experimenters sampled 512 new trajectories for a series of different p values. For p-greedy and p-quantiles, experimenters vary p between 0 and 1, for p-of-max the experimenters vary p between 0.9 and 1 (values below 0.9 have minor effects). In such cases, increasing p has the effect of increasing the average reward. Moreover, in such cases, the QGFN generation system experiences an increase in average reward without any retraining, even though the experimenters use values of p different than those utilized during training (e.g., p controls greediness). In other words, the QGFN generation system 100 can train the action-value function model with p-greedy QGFN and can further use the action-values with entirely different sampling strategies. As such, the QGFN generation system 100 can avoid training with high p values (e.g., which can reduce the diversity the model is exposed to) but from the training, the QGFN generation system 100 learns to sample new high-reward objects.

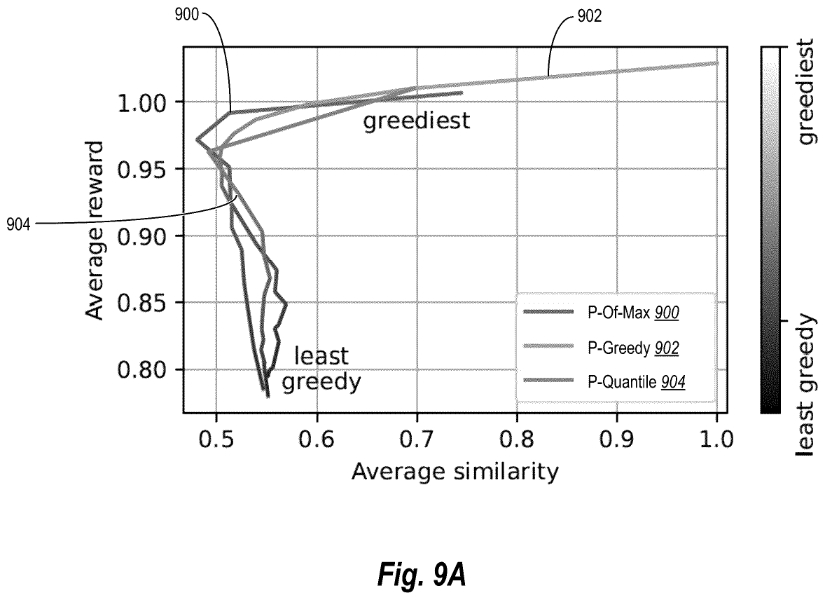

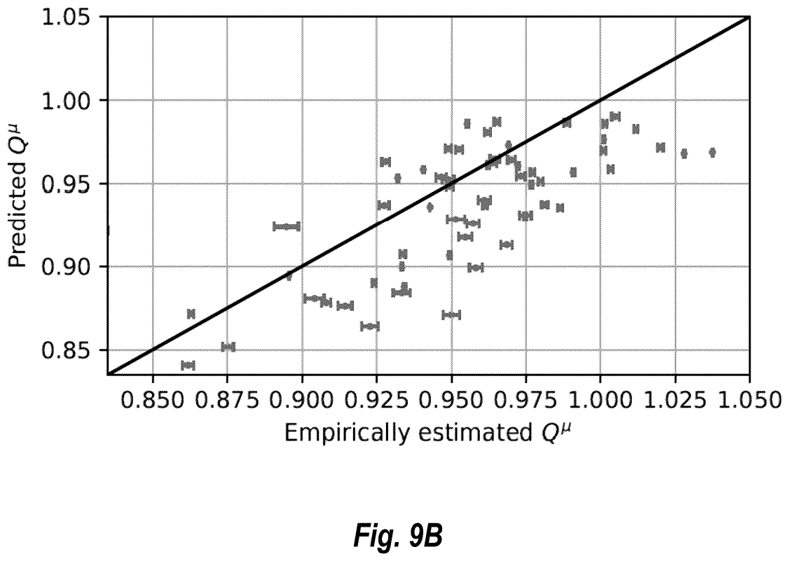

FIG. 9A illustrates that varying the p value at inference time induces reward-diversity trade-offs. Specifically, FIG. 9A illustrates a fragment-based molecule generation task, as the average similarity increases, the average reward similarly increases for all three methods (e.g., p-of-max QGFN 900, p-greedy QGFN 902, and p-quantile QGFN 904). In other words, FIG. 9A shows that for a low average similarity (e.g., ˜0.5), the p-of-max QGFN 900 shows the highest reward, the p-greedy QGFN 902 comes next, followed by the p-quantile QGFN 904.

FIG. 9B illustrates experimental results of the action-value function model compared with empirical estimates. Specifically, experimenters trained an action value function model and a generative flow network with a p-greedy QGFN method and a p value of 0.4 (e.g., for a fragment-based molecule task) and sampled n=64 trajectories. For instance, experimenters took random states within a trajectory as a starting point for m=512 trajectories, where the 512 trajectories have rewards as an empirical estimate Q of the expected return. In other words, Q should roughly predict the empirical estimates. FIG. 9B illustrates that the predicted Q (e.g., the action-value) for the behavior policy roughly tracks with the empirically estimated Q.

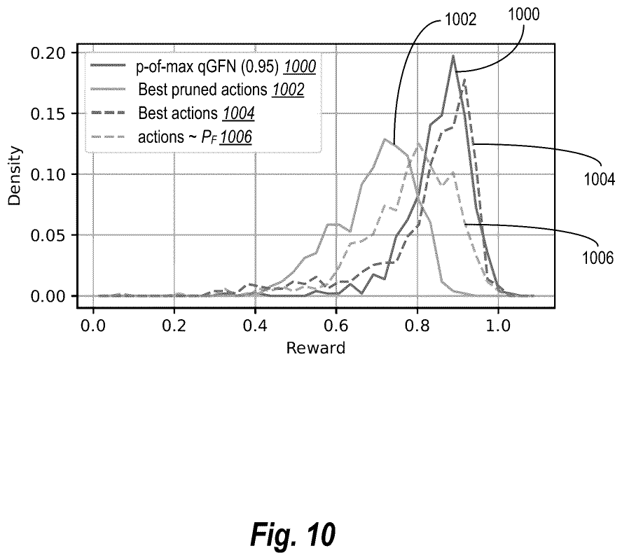

FIG. 10 illustrates that pruning (e.g., utilizing p-of-max QGFN) generally avoids low-reward parts of the state space. In other words, masking according to the action-value does not get rid of valuable flow measures. Specifically, FIG. 10 illustrates that pruning constructive object options based on the action-value is helpful. For example, experimenters utilized a trained model (QGFN) for the fragment-based molecular generation task and sampled n=512 trajectories. Further, experimenters utilized p-of-max QGFN 1000 (p=0.95) and compared it to the following i) best pruned actions 1002 for each trajectory (e.g., for each trajectory after a random number of steps, deterministically select the action most probable according to the forward policy flow measure that would be masked according to the action-value), ii) best actions 1004 (after some random number of trajectory steps (e.g., between 4 and 20), deterministically select the action that is most probable according to the forward policy flow measure, regardless of the action-value, and iii) the forward policy flow measure 1006.

As shown in FIG. 10, the best actions 1004 have little to no effect, while selecting actions that would have been pruned leads to much lower rewards (e.g., obviously selecting the actions based on the forward policy flow measure also leads to lower rewards). Thus, FIG. 10 confirms the hypothesis that the action-value indeed masks actions that are likely according to the forward policy but that do not consistently lead to high rewards.

In some embodiments, the QGFN generation system 100 prunes the action space based on the action-value, forming the basis for a constrained combinatorial optimization. In other words, the QGFN generation system 100 uses the action-value to predict some expected property or constraint, rather than reward. In doing so, the QGFN generation system 100 can prune some of the action space to avoid violating constraints or keeping some other properties below some threshold (e.g., synthesizability or toxicity in molecules).

The QGFN generation system 100 can utilize a variety of approaches for combining flow measures and action values. To illustrate, some additional methods of combining the action-value and the flow measure include a p-thresh approach (e.g., mask all action where Q(s, a)<p. Specifically, p-thresh includes closing off constructive object option paths where the action-value is less than the combination value (e.g., the p value which is a mixing hyperparameter). The QGFN generation system 100 can also utilize a soft-Q approach (e.g., as a baseline take softmax (Q/T) for some temperature T, which is varied as the greediness parameter). Specifically, soft-Q includes a variation where the QGFN generation system 100 takes the action-value divided by a temperature parameter, such that a low temperature parameter skews towards greedy actions and a high temperature parameter skews towards diversity.

The QGFN generation system 100 can also utilize a soft-Q [0.5] approach, where (Q/T) is mixed with the flow measure with a factor of the combination value being 0.5 (e.g., p=0.5). In other words, the QGFN generation system 100 utilizes a combination of 0.5 for the action-value and the flow measure but further adds in a temperature parameter. The QGFN generation system 100 can also utilize a GFN-then-Q approach. Specifically, for the first Np steps, the QGFN generation system 100 samples from forward policy flow measure, then sample greedily (where N is the maximum trajectory length. In other words, the QGFN generation system 100 can determine to start with prioritizing space exploration for the first Nₚ steps and switch to greedier actions after the first Nₚ steps. The QGFN generation system 100 can also utilize an MCTS approach (e.g., a Monte Carlo Tree Search where the forward policy flow measure is used as the expansion prior and maxₐ(Q(s, a) as the value of a state). In other words, during exploration, the QGFN generation system 100 prioritizes certain actions by referring to the flow measure and then evaluates the value of a state by considering the highest action-value achievable from that state.

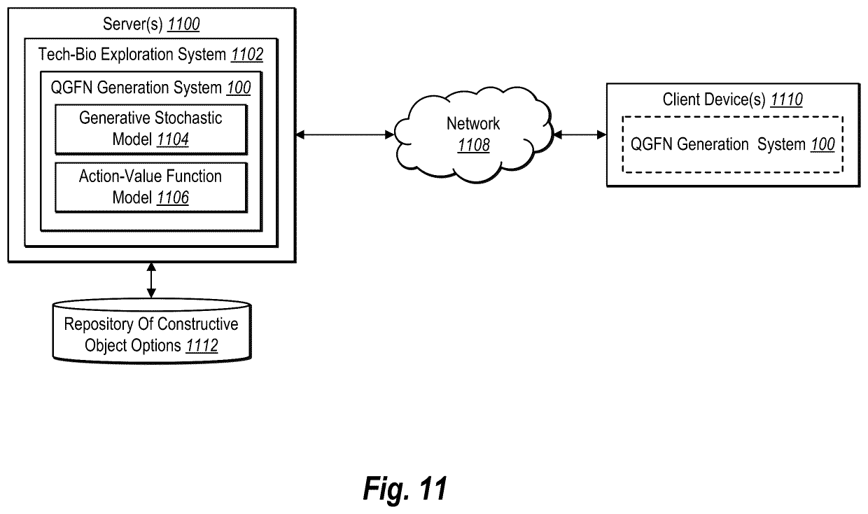

Additional detail regarding QGFN generation system 100 environment will now be provided with reference to FIG. 11. In particular, FIG. 11 illustrates a schematic diagram of a system environment in which the QGFN generation system 100 can operate in accordance with one or more embodiments.

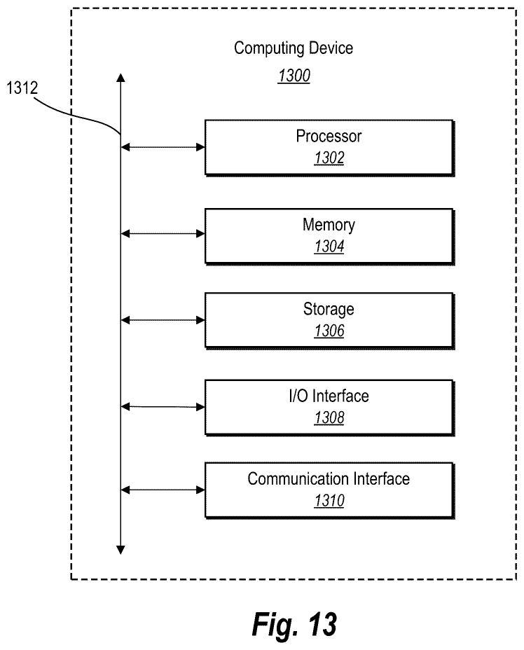

As shown in FIG. 11, the environment includes server(s) 1100 (which includes a tech-bio exploration system 1102 and the QGFN generation system 100), a network 1108, and client device(s) 1110. As further illustrated in FIG. 11, the various computing devices within the environment can communicate via the network 1108. Although FIG. 11 illustrates the QGFN generation system 100 being implemented by a particular component and/or device within the environment, the QGFN generation system 100 can be implemented, in whole or in part, by other computing devices and/or components in the environment (e.g., the additional device(s)). Additional description regarding the illustrated computing devices is provided with respect to FIG. 13 below.

As shown in FIG. 11, the server(s) 1100 (e.g., one or more local servers operated by a particular entity) can include the tech-bio exploration system 1102. In some embodiments, the tech-bio exploration system 1102 can determine, store, generate, and/or display tech-bio information including maps of biology, experiments from various sources, and/or machine learning tech-bio predictions. For instance, the tech-bio exploration system 1102 can analyze data signals corresponding to various treatments or interventions (e.g., compounds or biologics) and the corresponding relationships in genetics, proteomics, phenomics (i.e., cellular phenotypes), and invivomics (e.g., expressions or results within a living animal). Moreover, the tech-bio exploration system 1102 provides an environment for operating, executing, and managing complex drug discovery pipelines.

For instance, the tech-bio exploration system 1102 can generate and access experimental results corresponding to gene sequences, protein shapes/folding, protein/compound interactions, phenotypes resulting from various interventions or perturbations (e.g., gene knockout sequences or compound treatments), and/or invivo experimentation on various treatments in living animals. By analyzing these signals (e.g., utilizing various machine learning models), the tech-bio exploration system 1102 can generate or determine a variety of predictions and inter-relationships for improving treatments/interventions.

To illustrate, the tech-bio exploration system 1102 can generate maps of biology indicating biological inter-relationships or similarities between these various input signals to discover potential new treatments as part of the complex compound discovery process. For example, the tech-bio exploration system 1102 can utilize machine learning and/or maps of biology to identify a similarity between a first gene associated with disease treatment and a second gene previously unassociated with the disease based on a similarity in resulting phenotypes from gene knockout experiments. The tech-bio exploration system 1102 can then identify new treatments based on the gene similarity (e.g., by targeting compounds the impact the second gene). Similarly, the tech-bio exploration system 1102 can analyze signals from a variety of sources (e.g., protein interactions, or invivo experiments) to predict efficacious treatments based on various levels of biological data.

The tech-bio exploration system 1102 can generate GUIs comprising dynamic user interface elements to convey tech-bio information and receive user input for intelligently exploring tech-bio information. Indeed, as mentioned above, the tech-bio exploration system 1102 can generate GUIs displaying different maps of biology that intuitively and efficiently express complex interactions between different biological systems for identifying improved treatment solutions. Furthermore, the tech-bio exploration system 1102 can also electronically communicate tech-bio information between various computing devices.

As shown in FIG. 11, the tech-bio exploration system 1102 can include a system that facilitates various models or algorithms for generating maps of biology (e.g., maps or visualizations illustrating similarities or relationships between genes, proteins, diseases, compounds, and/or treatments) and discovering new treatment options over one or more networks. For example, the tech-bio exploration system 1102 collects, manages, and transmits data across a variety of different entities, accounts, and devices. In some cases, the tech-bio exploration system 1102 is a network system that facilitates access to (and analysis of) tech-bio information within a centralized operating system. Indeed, the tech-bio exploration system 1102 can link data from different network-based research institutions to generate and analyze maps of biology.

As shown in FIG. 11, the tech-bio exploration system 1102 can include a system that comprises the QGFN generation system 100 that generates, stores, manages, transmits data pertaining to biochemical structures built from a repository of constructive object options 1112. Specifically, FIG. 11 shows the QGFN generation system 100 further includes a generative stochastic model 1104 and an action-value function model 1106. For example, in context of the above description for the tech-bio exploration system 1102, in some embodiments the tech-bio exploration system 1102 further utilizes the QGFN generation system 100 to enhance the coordination between various groups involved in the drug discovery process. For instance, the QGFN generation system 100 works in tandem with the tech-bio exploration system 1102 to generate biochemical structures that indicate similarities or relationships between genes, proteins, diseases, compounds, and/or treatments) and can utilize generated biochemical structures to further discover new treatment options. Specifically, the tech-bio exploration system 1102 utilizes the QGFN generation system 100 to generate variations of different molecular structures based on different drug exploration objectives (e.g., generate a biochemical structure with a high binding affinity with a specific type of protein).

To illustrate, the QGFN generation system 100 utilizes the generative stochastic model 1104 and the action-value function model 1106 to select constructive object options from the repository of constructive object options 1112. Specifically, the QGFN generation system 100 determines to select one or more constructive object options based on the outputs from the generative stochastic model 1104 and the action-value function model 1106. To further illustrate, the tech-bio exploration system 1102 utilizes the QGFN generation system 100 at the program discovery phase to identify compounds that target certain genes. For instance, the QGFN generation system 100 can test various hypotheses for how a gene is affected by a compound and utilizes the generative stochastic model 1104 to explore a large state space to efficiently learn active learning targets and utilizes the action-value function model 1106 to target high-reward actions. Moreover, in some embodiments, the tech-bio exploration system 1102 utilizes the QGFN generation system 100 at the hit-to-lead phase (e.g., where a set of feasible compounds have already been identified) and performs additional iterations of the feasible compounds to refine the set of feasible compounds (e.g., narrow down the list by exploring the state space and prioritizing greedier actions, as described above).

As also illustrated in FIG. 11, the environment includes the client device(s) 1110. As mentioned above, the client device(s) 1110 can be involved in the process of drug discovery. Thus, for example, the client device(s) 1110 can coordinate/manage generating a particular biochemical structure along with additional mode variations of the biochemical structure for further downstream testing. For instance, the client device(s) 1110 can coordinate/manage testing generated biochemical structures under various conditions to further determine whether to initiate one or more programs (industrial program generation or industrial compound generation) for one or more of the generated biochemical structures.

To illustrate, the client device(s) 1110 can include computing devices that implement or manage a compound program generation stage of a compound discovery process. Similarly, the client device(s) 1110 can include computing devices that implement or manage a compound lead generation stage and the client device(s) 1110 can include computing devices that implement or manage a compound/dose selection stage. For example, the QGFN generation system 100 can receive one or more requests to generate one or more biochemical structures according to an input state and an objective for that input state.

In some embodiments, the environment also includes additional device(s). For example, the QGFN generation system 100 can utilize the additional device(s) to further operate and manage downstream operations after generating one or more biochemical structures. For instance, the additional device(s) include experimental device(s) and analytical device(s). Further, in some instances, the additional device(s) also include the computing devices discussed below in FIG. 13.

Furthermore, in one or more implementations, the client device(s) 1110 include a client application. The client application can include instructions that (upon execution) cause the client device(s) 1110 to perform various actions. For example, a user of a user account can interact with the client application on the client device(s) 1110 to execute the generation of biochemical structures (e.g., or other non-biochemical structures) by exploring a state space and executing experiments or other multi-faceted based on generated biochemical structures. For instance, in some embodiments the QGFN generation system 100 receives a request to generate a biochemical structure from an input state and an objective for the input state. In response, the QGFN generation system 100 can further generate one or more biochemical structures according to the objective and returns the biochemical structure to the client device(s) 1110. In some instances, the transmittal of the biochemical structure to the client device(s) 1110 causes the client device(s) 1110 to further present options for executing an action (e.g., performing downstream experiments, tests, or evaluations of the generated biochemical structure).

Although not shown, the environment can also include dedicated training device(s). For example, the dedicated training device(s) can include computing devices or virtual machines dedicated to training or implementing the generative stochastic model 11-4 and the action-value function model 1106. For example, the dedicated training device(s) can provide datasets, parameters, objectives, and other learning constraints to train the generative stochastic model 1104 and the action-value function model 1106 to generate outputs specific to a task (e.g., fragment-based molecular generation, RNA generation, small molecule generation, etc.). Thus, the QGFN generation system 100 interacts with the dedicated training device(s) to learn certain state spaces and to accurately generate corresponding flow-measures and action-values.

The environment can also include experimental device(s). For example, the tech-bio exploration system 1102 can interact with the experimental device(s) that include intelligent robotic devices and camera devices for generating and capturing digital images of cellular phenotypes resulting from different perturbations (e.g., genetic knockouts or compound treatments of stem cells). Similarly, the experimental device(s) can include camera devices and/or other sensors (e.g., heat or motion sensors) capturing real-time information from animals as part of invivo experimentation. The tech-bio exploration system 1102 can also interact with a variety of other experimental device(s) such as devices for determining, generating, or extracting gene sequences or protein information. For example, the experimental device(s) may include computing devices linked to biosensorselectrophysiological platforms, x-ray crystallography machines, liquid chromatography mass spectrometry systems, nuclear magnetic resonance spectrometers, mass spectrometers. In some implementations, the QGFN generation system 100 generates the tractability scores and further determines to employ or utilize one or more experimental devices (e.g., to initiate one or more experiments based on the tractability scores).

As further shown in FIG. 11, the environment includes the network 1108. As mentioned above, the network 1108 can enable communication between components of the environment. In one or more embodiments, the network 1108 may include a suitable network and may communicate using a various number of communication platforms and technologies suitable for transmitting data and/or communication signals, examples of which are described with reference to FIG. 13. Furthermore, although FIG. 11 illustrates computing devices communicating via the network 1108, the various components of the environment can communicate and/or interact via other methods (e.g., communicate directly).

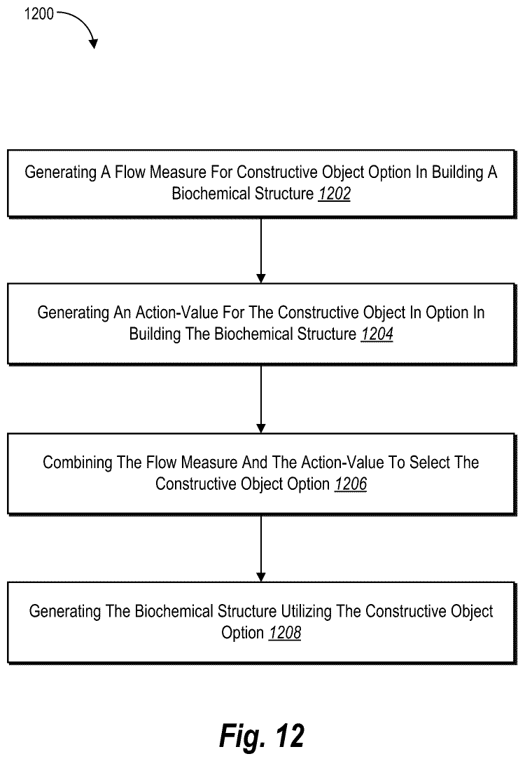

FIGS. 1-11, the corresponding text, and the examples provide a number of different systems, methods, and non-transitory computer readable media for utilizing a combination of a generative stochastic model and an action-value function model to generate a biochemical structure. In addition to the foregoing, embodiments can also be described in terms of flowcharts comprising acts for accomplishing a particular result. For example, FIG. 12 illustrates a flowchart of an example sequence of acts in accordance with one or more embodiments.

While FIG. 12 illustrates acts according to some embodiments, alternative embodiments may omit, add to, reorder, and/or modify any of the acts shown in FIG. 12. The acts of FIG. 12 can be performed as part of a method (e.g., a computer-implemented method). Alternatively, a non-transitory computer readable medium can comprise instructions, that when executed by one or more processors (e.g., at least one processor), cause a computing device to perform the acts of FIG. 12. In still further embodiments, a system can perform the acts of FIG. 12. Additionally, the acts described herein may be repeated or performed in parallel with one another or in parallel with different instances of the same or other similar acts.

FIG. 12 illustrates an example series of acts 1200 for generating a biochemical structure in accordance with one or more embodiments. The series of acts 1200 can include an act 1202 of generating a flow measure for constructive object option in building a biochemical structure, an act 1204 of generating an action-value for the constructive object option in building the biochemical structure, an act 1206 of combining the flow measure and the action-value to select the constructive object option, and an act 1208 of generating the biochemical structure utilizing the constructive object option. Specifically, the series of acts 1200 can include acts 1202-1208 of generating, utilizing a generative stochastic model, a flow measure for a constructive object option in building a biochemical structure; generating, utilizing an action-value function model, an action-value for the constructive object option in building the biochemical structure; combining the flow measure and the action-value to select the constructive object option from a plurality of constructive object options; and generating the biochemical structure utilizing the constructive object option

For example, in one or more embodiments, the series of acts 1200 includes generating a plurality of flow measures and a plurality of action-values for the plurality of constructive object options of a first construction stage from an input state of the biochemical structure. In one or more implementations, the series of acts 1200 includes utilizing the plurality of flow measures and the plurality of action-values to select the constructive object option for the first construction stage.

In addition, in one or more implementations, the series of acts 1200 includes generating an additional plurality of flow measures and an additional plurality of action-values for an additional plurality of constructive object options of a second construction stage from an additional input state of the biochemical structure; and selecting an additional constructive objection option utilizing the additional plurality of flow measures and the additional plurality of action-values.

Further, in some implementations, the series of acts 1200 includes wherein generating the flow measure for the constructive object option comprises generating a measure that indicates a cumulative probability of reward for downstream constructive object options based on selecting the constructive object option; and wherein generating the action-value for the constructive object option comprises generating a value that indicates an ultimate reward for selecting the constructive object option.

In one or more implementations, the series of acts 1200 includes generating an action-value flow measure that balances a cumulative reward indicated by the flow measure and an ultimate reward indicated by the action-value of the constructive object option according to a combination value. Moreover, in one or more implementations, the series of acts 1200 includes masking one or more constructive object options of the plurality of constructive object options by applying an action-value threshold to action-values corresponding to the one or more constructive object options; and selecting the constructive object option from the plurality of constructive object options based on the flow measure and the constructive object option not being masked.

In addition, in some implementations, the series of acts 1200 includes determining a plurality of flow measures and a plurality of action-values corresponding to the plurality of constructive object options; and selecting an action-value threshold based on the plurality of action-values.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 13 illustrates a block diagram of an example computing device 1300 that may be configured to perform one or more of the processes described above. One will appreciate that one or more computing devices, such as the computing device 1300 may represent the computing devices described above. In one or more embodiments, the computing device 1300 may be a mobile device (e.g., a mobile telephone, a smartphone, a PDA, a tablet, a laptop, a camera, a tracker, a watch, a wearable device, etc.). In some embodiments, the computing device 1300 may be a non-mobile device (e.g., a desktop computer or another type of client device). Further, the computing device 1300 may be a server device that includes cloud-based processing and storage capabilities.

As shown in FIG. 13, the computing device 1300 can include one or more processor(s) 1302, memory 1304, a storage device 1306, input/output interfaces 1308 (or “I/O interfaces 1308”), and a communication interface 1310, which may be communicatively coupled by way of a communication infrastructure (e.g., bus 1312). While the computing device 1300 is shown in FIG. 13, the components illustrated in FIG. 13 are not intended to be limiting. Additional or alternative components may be used in other embodiments. Furthermore, in certain embodiments, the computing device 1300 includes fewer components than those shown in FIG. 13. Components of the computing device 1300 shown in FIG. 13 will now be described in additional detail.

In particular embodiments, the processor(s) 1302 includes hardware for executing instructions, such as those making up a computer program. As an example, and not by way of limitation, to execute instructions, the processor(s) 1302 may retrieve (or fetch) the instructions from an internal register, an internal cache, memory 1304, or a storage device 1306 and decode and execute them.

The computing device 1300 includes memory 1304, which is coupled to the processor(s) 1302. The memory 1304 may be used for storing data, metadata, and programs for execution by the processor(s). The memory 1304 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 1304 may be internal or distributed memory.

The computing device 1300 includes a storage device 1306 includes storage for storing data or instructions. As an example, and not by way of limitation, the storage device 1306 can include a non-transitory storage medium described above. The storage device 1306 may include a hard disk drive (HDD), flash memory, a Universal Serial Bus (USB) drive or a combination these or other storage devices.

As shown, the computing device 1300 includes one or more I/O interfaces 1308, which are provided to allow a user to provide input to (such as user strokes), receive output from, and otherwise transfer data to and from the computing device 1300. These I/O interfaces 1308 may include a mouse, keypad or a keyboard, a touch screen, camera, optical scanner, network interface, modem, other known I/O devices or a combination of such I/O interfaces 1308. The touch screen may be activated with a stylus or a finger.

The I/O interfaces 1308 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 1308 are configured to provide graphical data to a display for presentation to a user. The graphical data may be representative of one or more graphical user interfaces and/or any other graphical content as may serve a particular implementation.

The computing device 1300 can further include a communication interface 1310. The communication interface 1310 can include hardware, software, or both. The communication interface 1310 provides one or more interfaces for communication (such as, for example, packet-based communication) between the computing device and one or more other computing devices or one or more networks. As an example, and not by way of limitation, communication interface 1310 may include a network interface controller (NIC) or network adapter for communicating with an Ethernet or other wire-based network or a wireless NIC (WNIC) or wireless adapter for communicating with a wireless network, such as a WI-FI. The computing device 1300 can further include a bus 1312. The bus 1312 can include hardware, software, or both that connects components of computing device 1300 to each other.

In one or more implementations, various computing devices can communicate over a computer network. This disclosure contemplates any suitable network. As an example, and not by way of limitation, one or more portions of a network may include an ad hoc network, an intranet, an extranet, a virtual private network (“VPN”), a local area network (“LAN”), a wireless LAN (“WLAN”), a wide area network (“WAN”), a wireless WAN (“WWAN”), a metropolitan area network (“MAN”), a portion of the Internet, a portion of the Public Switched Telephone Network (“PSTN”), a cellular telephone network, or a combination of two or more of these.

In particular embodiments, the computing device 1300 can include a client device that includes a requester application or a web browser, such as MICROSOFT INTERNET EXPLORER, GOOGLE CHROME, or MOZILLA FIREFOX, and may have one or more add-ons, plug-ins, or other extensions, such as TOOLBAR or YAHOO TOOLBAR. A user at the client device may enter a Uniform Resource Locator (“URL”) or other address directing the web browser to a particular server (such as server), and the web browser may generate a Hyper Text Transfer Protocol (“HTTP”) request and communicate the HTTP request to server. The server may accept the HTTP request and communicate to the client device one or more Hyper Text Markup Language (“HTML”) files responsive to the HTTP request. The client device may render a webpage based on the HTML files from the server for presentation to the user. This disclosure contemplates any suitable webpage files. As an example, and not by way of limitation, webpages may render from HTML files, Extensible Hyper Text Markup Language (“XHTML”) files, or Extensible Markup Language (“XML”) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a webpage encompasses one or more corresponding webpage files (which a browser may use to render the webpage) and vice versa, where appropriate.

In particular embodiments, the tech-bio exploration system 1102 may include a variety of servers, sub-systems, programs, modules, logs, and data stores. In particular embodiments, the tech-bio exploration system 1102 may include one or more of the following: a web server, action logger, API-request server, transaction engine, cross-institution network interface manager, notification controller, action log, third-party-content-object-exposure log, inference module, authorization/privacy server, search module, user-interface module, user-profile (e.g., provider profile or requester profile) store, connection store, third-party content store, or location store. The tech-bio exploration system 1102 may also include suitable components such as network interfaces, security mechanisms, load balancers, failover servers, management-and-network-operations consoles, other suitable components, or any suitable combination thereof. In particular embodiments, the tech-bio exploration system 1102 may include one or more user-profile stores for storing user profiles and/or account information for credit accounts, secured accounts, secondary accounts, and other affiliated financial networking system accounts. A user profile may include, for example, biographic information, demographic information, financial information, behavioral information, social information, or other types of descriptive information, such as interests, affinities, or location.

The web server may include a mail server or other messaging functionality for receiving and routing messages between the tech-bio exploration system 1102 and one or more client devices. An action logger may be used to receive communications from a web server about a user's actions on or off the tech-bio exploration system 1102. In conjunction with the action log, a third-party-content-object log may be maintained of user exposures to third-party-content objects. A notification controller may provide information regarding content objects to a client device. Information may be pushed to a client device as notifications, or information may be pulled from a client device responsive to a request received from the client device. Authorization servers may be used to enforce one or more privacy settings of the users of the tech-bio exploration system 1102. A privacy setting of a user determines how particular information associated with a user can be shared. The authorization server may allow users to opt in to or opt out of having their actions logged by the tech-bio exploration system 1102 or shared with other systems, such as, for example, by setting appropriate privacy settings. Third-party-content-object stores may be used to store content objects received from third parties. Location stores may be used for storing location information received from a client device associated with users.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A computer-implemented method comprising:
generating, utilizing a generative stochastic model, a flow measure for a constructive object option in building a biochemical structure;
generating, utilizing an action-value function model, an action-value for the constructive object option in building the biochemical structure;
combining the flow measure and the action-value to select the constructive object option from a plurality of constructive object options; and
generating the biochemical structure utilizing the constructive object option.

2. The computer-implemented method of claim 1, wherein generating the flow measure and generating the action-value comprises generating a plurality of flow measures and a plurality of action-values for the plurality of constructive object options of a first construction stage from an input state of the biochemical structure.

3. The computer-implemented method of claim 2, wherein combining the flow measure and the action-value comprises utilizing the plurality of flow measures and the plurality of action-values to select the constructive object option for the first construction stage.

4. The computer-implemented method of claim 3, further comprising:
generating an additional plurality of flow measures and an additional plurality of action-values for an additional plurality of constructive object options of a second construction stage from an additional input state of the biochemical structure; and
selecting an additional constructive objection option utilizing the additional plurality of flow measures and the additional plurality of action-values.

5. The computer-implemented method of claim 1,
wherein generating the flow measure for the constructive object option comprises generating a measure that indicates a cumulative probability of reward for downstream constructive object options based on selecting the constructive object option; and
wherein generating the action-value for the constructive object option comprises generating a value that indicates an ultimate reward for selecting the constructive object option.

6. The computer-implemented method of claim 1, wherein combining the flow measure and the action-value comprises generating an action-value flow measure that balances a cumulative reward indicated by the flow measure and an ultimate reward indicated by the action-value of the constructive object option according to a combination value.

7. The computer-implemented method of claim 1, wherein combining the flow measure and the action-value comprises:
masking one or more constructive object options of the plurality of constructive object options by applying an action-value threshold to action-values corresponding to the one or more constructive object options; and
selecting the constructive object option from the plurality of constructive object options based on the flow measure and the constructive object option not being masked.

8. The computer-implemented method of claim 1, wherein combining the flow measure and the action-value comprises:
determining a plurality of flow measures and a plurality of action-values corresponding to the plurality of constructive object options; and
selecting an action-value threshold based on the plurality of action-values.

9. A system comprising:
at least one processor; and
at least one non-transitory computer-readable storage medium storing instructions that, when executed by the at least one processor, cause the system to:
generate, utilizing a generative stochastic model, a flow measure for a constructive object option in building a biochemical structure;
generate, utilizing an action-value function model, an action-value for the constructive object option in building the biochemical structure;
combine the flow measure and the action-value to select the constructive object option from a plurality of constructive object options; and
generate the biochemical structure utilizing the constructive object option.

10. The system of claim 9, further comprising instructions that, when executed by the at least one processor, cause the system to generate a plurality of flow measures and a plurality of action-values for the plurality of constructive object options of a first construction stage from an input state of the biochemical structure.

11. The system of claim 10, further comprising instructions that, when executed by the at least one processor, cause the system to utilize the plurality of flow measures and the plurality of action-values to select the constructive object option for the first construction stage.

12. The system of claim 11, further comprising instructions that, when executed by the at least one processor, cause the system to:
generate an additional plurality of flow measures and an additional plurality of action-values for an additional plurality of constructive object options of a second construction stage from an additional input state of the biochemical structure; and
select an additional constructive objection option utilizing the additional plurality of flow measures and the additional plurality of action-values.

13. The system of claim 9, further comprising instructions that, when executed by the at least one processor, cause the system to:
generate the flow measure for the constructive object option by generating a measure that indicates a cumulative probability of reward for downstream constructive objective options based on selecting the constructive object option; and
generating the action-value for the constructive object option by generating a value that indicates an ultimate reward for selecting the constructive object option.

14. The system of claim 9, further comprising instructions that, when executed by the at least one processor, cause the system to combine the flow measure and the action-value by generating an action-value flow measure that balances a cumulative reward indicated by the flow measure and an ultimate reward indicated by the action-value of the constructive object option according to a combination value.

15. The system of claim 9, further comprising instructions that, when executed by the at least one processor, cause the system to combine the flow measure and the action-value by:
masking one or more constructive object options of the plurality of constructive object options by applying an action-value threshold to action-values corresponding to the one or more constructive object options; and
selecting the constructive object option from the plurality of constructive object options based on the flow measure and the constructive object option not being masked.

16. A non-transitory computer-readable medium storing instructions that, when executed by at least one processor, cause a computing device to:
generate, utilizing a generative stochastic model, a flow measure for a constructive object option in building a biochemical structure;
generate, utilizing an action-value function model, an action-value for the constructive object option in building the biochemical structure;
combine the flow measure and the action-value to select the constructive object option from a plurality of constructive object options; and
generate the biochemical structure utilizing the constructive object option.

17. The non-transitory computer-readable medium of claim 16, further comprising instructions that, when executed by the at least one processor, cause the computing device to generate a plurality of flow measures and a plurality of action-values for the plurality of constructive object options of a first construction stage from an input state of the biochemical structure.

18. The non-transitory computer-readable medium of claim 17, further comprising instructions that, when executed by the at least one processor, cause the computing device to utilize the plurality of flow measures and the plurality of action-values to select the constructive object option for the first construction stage.

19. The non-transitory computer-readable medium of claim 18, further comprising instructions that, when executed by the at least one processor, cause the computing device to:
generate the flow measure for the constructive object option by generating a measure that indicates a cumulative probability of reward for downstream constructive objective options based on selecting the constructive object option; and
generating the action-value for the constructive object option by generating a value that indicates an ultimate reward for selecting the constructive object option.

20. The non-transitory computer-readable medium of claim 16, further comprising instructions that, when executed by the at least one processor, cause the computing device to combine the flow measure and the action-value by generating an action-value flow measure that balances a cumulative reward indicated by the flow measure and an ultimate reward indicated by the action-value of the constructive object option according to a combination value.
